(19) United States
(12) Patent Application Publication
(10) Pub. No.: US 2021/0256872 A1
(43) Pub. Date: Aug. 19, 2021

(54) DEVICES, SYSTEMS, AND METHODS FOR PREDICTING BLOOD GLUCOSE LEVELS BASED ON A PERSONALIZED BLOOD GLUCOSE REGULATION MODEL

(71) Applicant: GlucoGear Tecnologia LTDA, Ribeirão Preto (BR)

(72) Inventors: Yuri Kendji Matsumoto, São Paulo (BR); Eduardo De Matos Rodrigues, São Paulo (BR); Vitor Pucciarelli Antloga, São Paulo (BR); Gabriel De Souza Pereira Moreira, São Paulo (BR); Camilo Rodrigues Neto, São Paulo (BR); Maria Cristina Foss Foss-Freitas, São Paulo (BR)

(73) Assignee: GlucoGear Tecnologia LTDA, Ribeirão Preto (BR)

(21) Appl. No.: 17/177,635

(22) Filed: Feb. 17, 2021

Related U.S. Application Data
(60) Provisional application No. 62/977,536, filed on Feb. 17, 2020.

(51) Int. Cl. G09B 19/00; A61B 5/00; B60Q 9/00; G06N 20/00; G16H 10/60; G16H 50/50; G16H 20/60; G16H 50/20

(52) U.S. Cl. CPC G09B 19/0092; A61B 5/14532; A61B 5/4866; A61B 5/7275; A61B 5/7475; A61B 5/746; A61B 5/7267; A61B 5/4848; B60Q 9/00; G06N 20/00; G16H 10/60; G16H 50/50; G16H 20/60; G16H 50/20; A61B 5/486

(57) ABSTRACT

The present disclosure provides devices, systems, and methods for optimizing blood glucose level regulation by predicting blood glucose levels based on personalized blood glucose regulation models. In some exemplary embodiments, a computer-implemented method of blood glucose level regulation includes collecting a first plurality of data sets associated with an individual from a database, generating a personalized blood glucose regulation model for the individual, receiving a second plurality of data sets associated with the individual, and generating predicted blood glucose levels for the individual using the personalized blood glucose regulation model and the second plurality of data sets. The personalized blood glucose regulation model can also be used to identify risks of blood glucose excursions, titrate insulin doses, optimize nutritional and physical activities plans, recommend preventive and corrective actions, and send signals, such as alerts and commands, to insulin delivery devices or other devices or systems to improve safety of the individual.

### CROSS-REFERENCE TO RELATED APPLICATION

This application claims the benefit of priority of U.S. Provisional Patent Application No. 62/977,536, filed Feb. 17, 2020, which is incorporated herein by reference.

### TECHNICAL FIELD

The present disclosure relates to devices, systems, and methods for predicting blood glucose levels based on personalized blood glucose regulation models and corresponding uses of the devices, systems, and methods for blood glucose regulation.

### BACKGROUND

Metabolic disorders are characterized by the physiological deficiency in the metabolism process to supply body cells with energy, which leads to complications and adverse effects, such as weight gain or loss. Diabetes is one of the main metabolic disorders globally and has two main types: type 1 diabetes (T1D) and type 2 diabetes (T2D). T1D is an autoimmune disease that causes the deficiency in secretion of insulin, a hormone acting on glucose permeability in body cells and affecting blood glucose levels (BGL). Fluctuations in BGL are normal and happen daily in people without diabetes. As shown in FIG. 1, a normal range of blood sugar concentration is typically between about 3.9 and about 7.8 mmol/L. A hyperglycemic state occurs, or hyperglycemic excursions occur, when blood sugar levels are higher than the normal range. A hypoglycemic state occurs, or hypoglycemic excursions occur, when blood sugar levels are lower than the normal range. T1D can cause a hyperglycemic state and requires daily insulin injections to supply body cells with energy and to avoid complications related to hyperglycemia, such as retinopathy, neuropathy, and vasculopathy. T2D is characterized by the decrease in insulin sensitivity, which leads to the same hyperglycemic state. For a patient with T2D, although the pancreas still secretes insulin, the patient may still require exogenous insulin injections in addition to antidiabetic drugs, nutritional and physical activity changes improve BGL regulation.

Conventional methods or protocols for regulating BGL of an individual having T1D involve injecting a bolus insulin (regular or fast-acting) to compensate or correct postprandial hyperglycemia caused by carbohydrates intake. Sometimes, the dose of the bolus insulin is calculated based on carbohydrate counting and insulin-to carb ratio. In other instances, insulin doses are kept fixed and the amount of carbohydrate that should be eaten at each meal (e.g., 10 units at breakfast, 20 units at lunch, 10 units at dinner) is regulated. Such method specifies the bolus dosing for prandial correction based only on the amount of carbohydrates the individual takes to simplify the dosing calculation by focusing on the macronutrient that causes the highest hyperglycemic effect. This simple method can be used with minimal instruction and thus is commonly being used by physicians and patients. However, insulin therapy may cause hypoglycemia if the injected insulin doses are higher than necessary and can also lead to severe adverse effects such as fainting, coma, or seizures. Therefore, it's important to use suitable doses of insulin to regulate blood glucose.

The simple method discussed above neglects many other factors involved in the complex process of BGL regulation. For example, different nutrients can have different effects on BGL after each meal. FIG. 2 is a graphical representation of exemplary blood glucose profiles after intake of carbohydrates, proteins, and fat or lipids. As shown in FIG. 2, different types of nutrients can cause BGL to increase at different rates, resulting in different blood glucose profiles. For example, carbohydrates typically cause a BGL peak in about 15-30 minutes, proteins typically cause a BGL peak in about 2-3 hours, and fats or lipids typically cause a BGL peak in about 1-3 hours with longer duration in glucose intake curve. Additionally, different types of carbohydrates can cause different blood glucose profiles. As shown in FIG. 2, simple carbohydrates typically cause a BGL peak sooner than complex carbohydrates do. Physical activity of a patient can change the patient's sensitivity to insulin intake, which can directly affect the glucose regulation of an insulin dosage.

The bolus insulin (regular or fast-acting bolus insulin) used in the method discussed above can only correct the hyperglycemic effect caused by carbohydrates, e.g., correcting the BGL peak around 15-45 minutes after a meal, but do not correct the BGL peaks caused by proteins and lipids, which can lead to a rebound effect about 1-6 hours after a meal containing large amounts of proteins and lipids (e.g., burgers, barbecue, etc.). Additionally, fiber intake can alter the blood glucose profile of carbohydrates, e.g., white bread and multi-grain bread have different blood glucose profiles. Failing to consider the different effects of proteins, lipids, and fibers on glucose adsorption rates and blood glucose profiles in the current methods for BGL regulation has become even more problematic lately due to the adoption of new diets, such as low-carb diets with higher fiber intake, Paleo diets, and keto diets. Using the current methods for regulating BGL can lead to inaccuracy in insulin dosing patients adopting the new diets and, consequently, poor blood glucose regulation.

Another deficiency of the current methods for BGL regulation is that physicians usually determine the initial insulin dosing regimen using generic standard protocols that are based on some factors, such as weight, age, and sex. The physicians then correct the insulin dosing regimen empirically from consultation to consultation, based on physiological changes during puberty, pregnancy and medical illness, which can be three to six months apart. However, such protocols do not account for each individual's unique metabolic pattern and this metabolic pattern changes over time according to the individual's different physiological conditions, such as during menses, pregnancy, or infections. Some studies have found that the overall incidence rate of hypoglycemia is high, causing substantial impact on both productivity and health care utilization, with large variations between patient cohorts, thus reinforcing the need to create better methods to regulate BGL. Additionally, the current methods require that the physicians speculate on an initial prescription and adjust insulin dosing empirically from consultation to consultation. This can cause long periods of time during which a patient is not optimally dosed and with glucose levels not properly regulated. For patients that engage in physical activities, the current methods are even more problematic.

In short, the current methods for BGL regulation based on carbohydrate counting and insulin-to carb ratio are deficient to achieve optimal BGL regulation for most patients. Furthermore, it is difficult for physicians to consider the factors that can affect BGL in each patient, such as the intake of proteins, lipids, fibers, and the engagement of physical activity of each patient, and empirically determine insulin dosing regimens for each patient in a timely manner. Therefore, systems, devices, and methods are needed to allow physicians to have faster and more accurate understanding of the glucose metabolic pattern of each patient and to determine insulin prescription suitable for that patient. Additionally, systems, devices, and methods are needed to allow patients to be advised on more accurate insulin dosing more frequently and, ideally, at least daily.

U.S. Pat. No. 10,169,544 discloses a glucose dynamics simulator for T1D based on real data from a clinical trial with T1D human subjects. However, it describes a standard and generic mathematical model to simulate glucose regulation dynamics in T1D and does not provide a personalized solution for each individual.

WO 2018/229209 discloses a method for predicting hypoglycemia and adjusting insulin doses for a subject using an insulin syringe pen for multi-daily injections (MDI). However, the method considers only the effect of carbohydrates on BGL but neglects the effect of other relevant nutrients, such as proteins, lipids, and fibers, or the effect of physical activities, which change blood glucose regulation during and after the activity.

WO 2018/145965 discloses an alert system for hypoglycemia events while a patient is driving a vehicle. The alert system uses a statistical and probability approach to identify hypoglycemia risk. However, the statistical and probability approach does not provide good results to predict changes in the direction of the blood glucose profile and may cause failures to predict hypoglycemia after the injection of various fast or ultra-fast acting insulin.

None of the aforementioned methods or systems solve the above-discussed problems.

### SUMMARY

The present disclosure describes devices, systems, and methods for generating a personalized blood glucose regulation model for an individual based on data and signals of the individual. The personalized blood glucose regulation model can be used in various applications to support individuals with metabolic disorders, such as people with diabetes (PWD) or any individual wanting to regulate or maintain blood glucose level, and to assist health care professionals in providing personalized blood glucose regulation optimization strategies and safety actions for an individual. For example, the personalized blood glucose regulation model can be used to predict blood glucose levels, identify risks of blood glucose excursions, titrate insulin doses, optimize nutritional and physical activities plans, recommend preventive and corrective actions, send signals, such as alerts and commands, to insulin delivery devices.

PWD are subject to a wide variety of driving requirements and restrictions, as hypoglycemia can cause change or loss of judgment and awareness, thereby increasing driving mishaps. Although diabetes-related traffic accidents are relatively infrequent for drivers with diabetes, technologies that allow sending glycemic excursions alerts to the driver are still beneficial to reduce or avoid driving mishaps. Such technologies would also provide benefits for PWD operating machinery or other equipment including airplanes, where diabetes-related maloperation could lead to serious accidents. In some embodiments, the personalized blood glucose regulation model can be used to send signals, such as alerts and commands, to driving systems, vehicles, machines, or equipment to reduce or prevent potential accidents.

According to an exemplary embodiment of the present disclosure, a computer-implemented method of optimizing blood glucose level regulation is provided. In some embodiments, the method of optimizing blood glucose level regulation includes collecting a first plurality of data sets associated with an individual from a database. In some embodiments, the first plurality of data sets includes nutritional intake data at a plurality of time points over a first period. In some embodiments, the method of optimizing blood glucose level regulation includes a method for generating a personalized blood glucose regulation model for the individual. In some embodiments, the method of optimizing blood glucose level regulation includes receiving a second plurality of data sets associated with the individual. In some embodiments, the second plurality of data sets includes nutritional intake data at a plurality of time points over a second period. In some embodiments, the method of optimizing blood glucose level regulation further includes generating predicted blood glucose levels at one or more time points after the second period using the personalized blood glucose regulation model and the second plurality of data sets. In some embodiments, the method of optimizing blood glucose level regulation further includes providing an instruction for regulating an individual's blood glucose level based on the generated predicted blood glucose levels.

In some embodiments, the instruction for regulating an individual's blood glucose level is an instruction for taking a dose of bolus and/or basal insulin. In some embodiments, the instruction for regulating an individual's blood glucose level is an instruction for performing a personalized blood glucose regulation optimization strategy (e.g., a bolus and/or basal insulin titration or therapy, a nutritional plan, or a physical activity plan). In some embodiments, the instruction for regulating an individual's blood glucose level is an instruction for taking a personalized preventive or corrective action for the individual.

According to an exemplary embodiment of the present disclosure, a non-transitory computer-readable storage medium is provided. The non-transitory computer-readable storage medium stores a set of instructions that, when executed by one or more processors, cause the one or more processors to perform a method of optimizing blood glucose level regulation. In some embodiments, the method of optimizing blood glucose level regulation includes collecting a first plurality of data sets associated with an individual from a database. In some embodiments, the first plurality of data sets includes nutritional intake data at a plurality of time points over a first period. In some embodiments, the method of optimizing blood glucose level regulation includes a method for generating a personalized blood glucose regulation model for the individual. In some embodiments, the method of optimizing blood glucose level regulation includes receiving a second plurality of data sets associated with the individual. In some embodiments, the second plurality of data sets includes nutritional intake data at a plurality of time points over a second period. In some embodiments, the method of optimizing blood glucose level regulation further includes generating predicted blood glucose levels at one or more time points after the second period using the working personalized blood glucose regulation model and the second plurality of data sets. The method of optimizing blood glucose level regulation may further include providing an instruction for regulating the individual's blood glucose level based on the generated predicted blood glucose levels.

In some embodiments, the nutritional intake data include carbohydrate intake, protein intake, and lipid intake. In some embodiments, the nutritional intake data include blood glucose level measurements at a plurality of time points over the first period. In some embodiments, the nutritional intake data include insulin intake data at a plurality of time points over the first period. In some embodiments, the nutritional intake data of the second plurality of data sets includes at least one of carbohydrate intake, protein intake, or lipid intake. In some embodiments, the second plurality of data sets includes insulin intake data over the second period.

According to an exemplary embodiment of the present disclosure, a computer-implemented method of optimizing blood glucose level regulation is provided. In some embodiments, the method of optimizing blood glucose level regulation includes receiving a personalized blood glucose regulation model for an individual from a remote server over a network. In some embodiments, the remote server includes a non-transitory computer-readable storage medium storing a set of instructions that, when executed by the remote server, cause the remote server to perform a method for generating the personalized blood glucose regulation model.

In some embodiments, the method for generating a personalized blood glucose regulation model for the individual includes creating a first training data subset, a first validation data subset, and a first test data subset from the first plurality of data sets. In some embodiments, the method for generating a personalized blood glucose regulation model for the individual includes determining a plurality of parameters of the personalized blood glucose regulation model using an optimization algorithm, the first training data subset, and the first validation data subset. In some embodiments, the method for generating a personalized blood glucose regulation model for the individual includes determining a first evaluation metric of the personalized blood glucose regulation model using the first test data subset. In some embodiments, the method for generating a personalized blood glucose regulation model for the individual includes determining an approved personalized blood glucose regulation model based on the first evaluation metric. In some embodiments, the method for generating a personalized blood glucose regulation model for the individual includes determining a working personalized blood glucose regulation model based on the approved personalized blood glucose regulation model.

In some embodiments, the method for generating a personalized blood glucose regulation model for the individual includes collecting a third plurality of data sets associated with a group of individuals sharing one or more characteristics from the database. In some embodiments, the third plurality of data sets includes nutritional intake data at a plurality of time points over the first period. In some embodiments, the nutritional intake data comprising at least one of carbohydrate intake, protein intake, or lipid intake. In some embodiments, the third plurality of data sets includes insulin intake data at a plurality of time points over the first period. In some embodiments, the method for generating a personalized blood glucose regulation model for the individual includes generating a populational blood glucose regulation base model. In some embodiments, generating the populational blood glucose regulation base model includes creating a second training data subset, a second validation data subset, and a second test data subset based on the third plurality of data sets. In some embodiments, generating the populational blood glucose regulation base model includes determining a plurality of parameters of the populational blood glucose regulation base model using an optimization algorithm, the second training data subset, and the second validation data subset. In some embodiments, generating the populational blood glucose regulation base model includes determining a second evaluation metric of the populational blood glucose regulation base model using the second test data subset. In some embodiments, generating the populational blood glucose regulation base model includes determining an approved populational blood glucose regulation base model based on the second evaluation metric. In some embodiments, the method for generating a personalized blood glucose regulation model for the individual includes determining a third evaluation metric for the approved populational blood glucose regulation base model using the first test data subset. In some embodiments, the method for generating a personalized blood glucose regulation model for the individual includes determining the personalized blood glucose regulation model or the populational blood glucose regulation base model as the working personalized blood glucose regulation model based on the second and third evaluation metrics.

In some embodiments, the method for generating a personalized blood glucose regulation model for the individual includes collecting a third plurality of data sets associated with the individual from the database. In some embodiments, the third plurality of data sets includes nutritional intake data at a plurality of time points over a third period. In some embodiments, the nutritional intake data comprising carbohydrate intake, protein intake, and lipid intake. In some embodiments, the third plurality of data sets includes blood glucose level measurements at a plurality of time points over the third period. In some embodiments, the third plurality of data sets includes insulin intake data at a plurality of time points over the third period. In some embodiments, the method for generating a personalized blood glucose regulation model for the individual includes generating predicted blood glucose levels at a plurality of time points over a first-time interval of the third period using the personalized blood glucose regulation model and the third plurality of data sets. In some embodiments, the method for generating a personalized blood glucose regulation model for the individual includes creating a second training data set, a second validation data set, and a second test data set using the third plurality of data sets and the predicted blood glucose levels at the plurality of time points over the first-time interval. In some embodiments, the method for generating a personalized blood glucose regulation model for the individual includes training a machine learning model using the second training data set and the second validation data set. In some embodiments, the method for generating a personalized blood glucose regulation model for the individual includes determining a second evaluation metric of the machine learning model using the second test data set. In some embodiments, the method for generating a personalized blood glucose regulation model for the individual includes generating a personalized hybrid model including the personalized blood glucose regulation model and the machine learning model. In some embodiments, the method for generating a personalized blood glucose regulation model for the individual includes determining the personalized hybrid model as the working personalized blood glucose regulation model.

Additional disclosure of the disclosed embodiments will be set forth in part in the description that follows.

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain exemplary principles of certain disclosed embodiments as set forth in the accompanying claims.

### BRIEF DESCRIPTION OF THE DRAWINGS

FIG. 1 is a graphical representation of exemplary blood glucose levels of a diabetic individual over time.

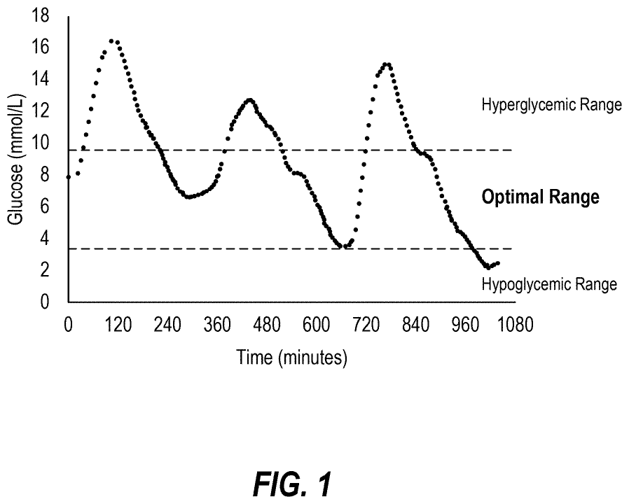

FIG. 2 is a graphical representation of exemplary blood glucose levels of an individual after eating certain nutrients.

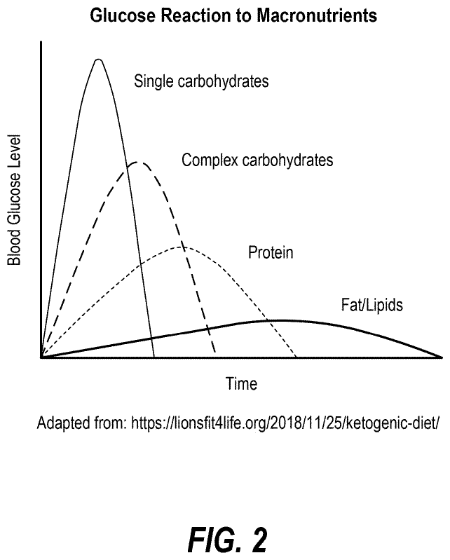

FIG. 3 is a schematic representation of an exemplary method of predicting blood glucose levels, according to some embodiments of the present disclosure.

FIG. 4 is a schematic representation of an exemplary process for compiling patient data, according to some embodiments of the present disclosure.

FIG. 5 is a schematic representation of an exemplary process for generating a personalized blood glucose regulation model, according to some embodiments of the present disclosure.

FIG. 6 is a schematic representation of an exemplary process for preprocessing compiled patient data for generating a personalized blood glucose regulation model, according to some embodiments of the present disclosure.

FIG. 7 is a schematic representation of an exemplary process for generating a populational blood glucose regulation base model, according to some embodiments of the present disclosure.

FIG. 8 is a schematic representation of an exemplary process for generating a hybrid personalized blood glucose regulation model, according to some embodiments of the present disclosure.

FIG. 9 is a schematic representation of an exemplary process for preprocessing compiled patient data for generating a populational blood glucose regulation base model, according to some embodiments of the present disclosure.

FIG. 10 is a schematic representation of an exemplary system for predicting blood glucose levels, according to some embodiments of the present disclosure.

FIG. 11 is a schematic representation of an exemplary service terminal for predicting blood glucose levels, according to some embodiments of the present disclosure.

FIG. 12 is a schematic representation of an exemplary user device for predicting blood glucose levels, according to some embodiments of the present disclosure.

FIG. 13 is a graphical representation of a user interface on an exemplary user device for predicting blood glucose levels, according to some embodiments of the present disclosure.

FIG. 14A is a graphical representation of simulated blood glucose levels and blood insulin levels of an exemplary diabetic individual after eating a meal with only carbohydrates without bolus insulin injection, according to some embodiments of the present disclosure.

FIG. 14B is a graphical representation of simulated blood glucose levels and blood insulin levels of the exemplary diabetic individual of FIG. 14A after eating the same meal with only carbohydrates and injecting a dose of bolus insulin, according to some embodiments of the present disclosure.

FIG. 15A is a graphical representation of simulated blood glucose levels and blood insulin levels of an exemplary diabetic individual after eating a meal containing carbohydrate, protein, and lipid without bolus insulin injection, according to some embodiments of the present disclosure.

FIG. 15B is a graphical representation of simulated blood glucose levels and blood insulin levels of an exemplary diabetic individual after eating the meal containing carbohydrate, protein, and lipid with injection of a dose of bolus insulin shortly before the meal, according to some embodiments of the present disclosure.

FIG. 15C is a graphical representation of simulated blood glucose levels and blood insulin levels of an exemplary diabetic individual after eating the meal containing carbohydrate, protein, and lipid with injection of a higher dose of bolus insulin shortly before the meal, according to some embodiments of the present disclosure.

FIG. 15D is a graphical representation of simulated blood glucose levels and blood insulin levels of the exemplary diabetic individual of FIG. 15A after eating the meal containing carbohydrate, protein, and lipid with the injection of two doses of bolus insulin at different times, according to some embodiments of the present disclosure.

FIG. 16 is a graphical representation of simulated blood glucose levels and blood insulin levels of an exemplary diabetic individual generated by an exemplary personalized blood glucose regulation model, according to some embodiments of the present disclosure.

FIG. 17A is a graphical representation of exemplary nutritional intake of an exemplary diabetic individual in a dawn period, according to some embodiments of the present disclosure.

FIG. 17B is a graphical representation of exemplary heart rate measurements of the exemplary diabetic individual of FIG. 17A in the same dawn period, according to some embodiments of the present disclosure.

FIG. 17C is a graphical representation of measured blood glucose levels and simulated blood glucose levels of the exemplary diabetic individual of FIG. 17A in the same drawn period.

FIG. 18A is a graphical representation of exemplary nutritional intake of an exemplary diabetic individual in a morning period, according to some embodiments of the present disclosure.

FIG. 18B is a graphical representation of exemplary heart rate measurements of the exemplary diabetic individual of FIG. 18A in the same morning period, according to some embodiments of the present disclosure.

FIG. 18C is a graphical representation of measured blood glucose levels and simulated blood glucose levels of the exemplary diabetic individual of FIG. 18A in the same morning period.

FIG. 19A is a graphical representation of exemplary nutritional intake of an exemplary diabetic individual in a night period, according to some embodiments of the present disclosure.

FIG. 19B is a graphical representation of exemplary heart rate measurements of the exemplary diabetic individual of FIG. 19A in the same night period, according to some embodiments of the present disclosure.

FIG. 19C is a graphical representation of measured blood glucose levels and simulated blood glucose levels of the exemplary diabetic individual of FIG. 19A in the same night period.

FIG. 20A is a graphical representation of exemplary nutritional intake of an exemplary diabetic individual in an afternoon period, according to some embodiments of the present disclosure.

FIG. 20B is a graphical representation of exemplary heart rate measurements of the exemplary diabetic individual of FIG. 20A in the same afternoon period, according to some embodiments of the present disclosure.

FIG. 20C is a graphical representation of measured blood glucose levels and simulated blood glucose levels of the exemplary diabetic individual of FIG. 20A in the same afternoon period.

FIG. 21 is a graphical representation of exemplary nutritional intake, insulin intake, measured blood glucose levels, and two exemplary simulated glucose curves of an exemplary diabetic individual, according to some embodiments of the present disclosure.

### DETAILED DESCRIPTION

Reference will now be made in detail to embodiments and aspects of the present disclosure, examples of which are illustrated in the accompanying drawings. Where possible, the same reference numbers will be used throughout the drawings to refer to the same or like parts.

The present disclosure provides devices, systems, and methods that allow for personalizing insulin therapy strategies for blood glucose control, such as, but not limited to, identifying risks and patterns of blood glucose excursions, titrate insulin doses, optimize nutritional and physical activities plans. Embodiments of the present disclosure allows for faster insulin dosing optimization (i.e., insulin titration). For example, in contrast to a physician starting the insulin treatment of a patient using the generic insulin titration method and then trying to optimize insulin dosing empirically after each consultation, which can lead to 6 months or even more than one year to adjust the insulin dosing, a personalized blood glucose regulation model mimicking the physiology of the patient can be created and used to provide better insulin dosing in the first or second consultation. In some embodiments, the personalized blood glucose regulation model can provide recommendations for insulin dosing to the patient daily or even more frequently, such as immediate recommendations upon the individual's request or data input. Embodiments described herein can create a personalized blood glucose regulation model to improve treatment strategies for T1D, T2D, or other metabolic disorders, and/or assist any individual with blood glucose regulation, weight control, or body muscle building. Although some figures and embodiments are described with respect to patients, a “patient” can be any individual wanting to regulate blood glucose levels.

In some embodiments, the present disclosure provides a technical solution for the problem associated with inaccurate BGL regulation of patients engaging in physical activities or under different physiological conditions. For example, embodiments of the present disclosure allow for creating a personalized blood glucose regulation model that accounts for physiological changes of a patient, such as physiological changes during an infection or menstruation, and using such personalized blood glucose regulation model to determine insulin dosing. In some embodiments, devices, systems, and methods of the present disclosure allow for automatic generation of a personalized blood glucose regulation model mimicking the blood glucose regulation of an individual based on various individual data or signals.

In some embodiments, such individual data or signals include one or more of blood glucose measurements, glucose target range, carbohydrate intake, protein intake, lipid intake, fiber intake, calorie intake, bolus insulin doses and type of insulin molecules previously taken, basal insulin doses type of insulin molecules previously taken, other types of medicine previously taken, physical activity data, patient profile data, and body index data of an individual. In some embodiments, the physical activity data include one or more of heart rate measurements, calories or kilocalories burned, steps, type of activity, duration of activity, Metabolic Equivalent of Task (MET), duration of sleep, and phase of sleep. In some embodiments, the patient profile data includes one or more of sex, birth date, type of diabetes, target glucose range, weight, height, types of insulin molecules, and drug therapy. In some embodiments, the body index data includes at least one of weight, height, or body mass index (BMI).

In some embodiments, the personalized blood glucose regulation model is created by an automated process. In some embodiments, the personalized blood glucose regulation model is created using machine learning techniques to generate a more accurate personalized blood glucose regulation model and BGL predictions for an individual.

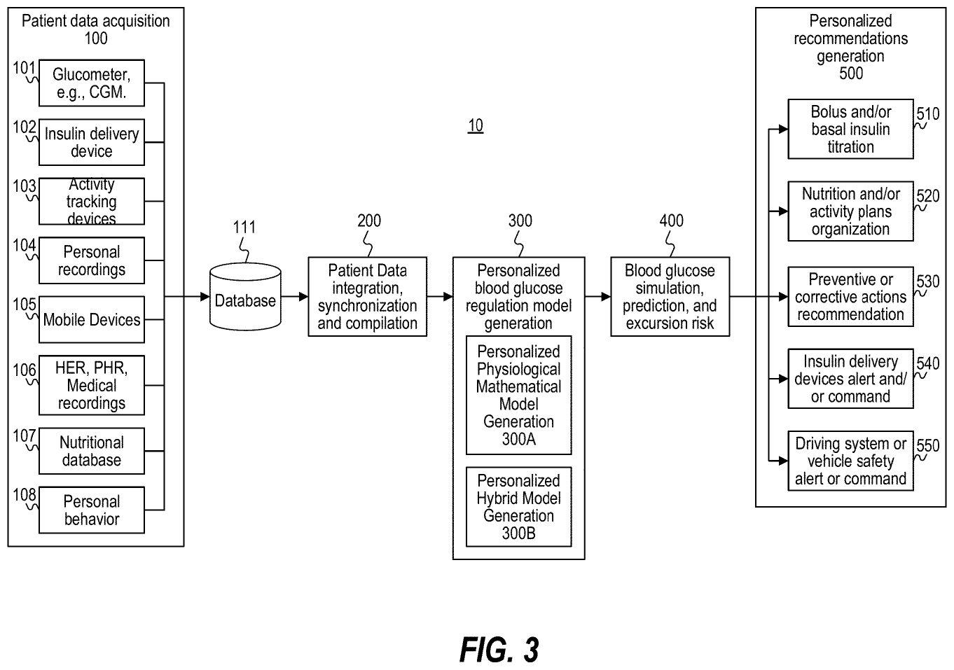

FIG. 3 is a schematic representation of an exemplary method 10 of predicting blood glucose levels, according to some embodiments of the present disclosure. In some embodiments, method 10 includes a plurality of processes, including a patient data acquisition process 100, a patient data compilation process 200, a personalized blood glucose regulation model generation process 300, and a blood glucose level prediction process 400. Each process of method 10 may include one or more operations. In some embodiments, as shown in FIG. 3, patient data compilation process 200 may include multiple operations, such as patient data integration, synchronization, and compilation. In some embodiments, a personalized blood glucose regulation model generation process 300 may perform one or more processes among a plurality of processes to determine a personalized blood glucose regulation model. In some embodiments, blood glucose level prediction process 400 may include blood glucose level simulation and/or prediction of risks of excursions of BGL.

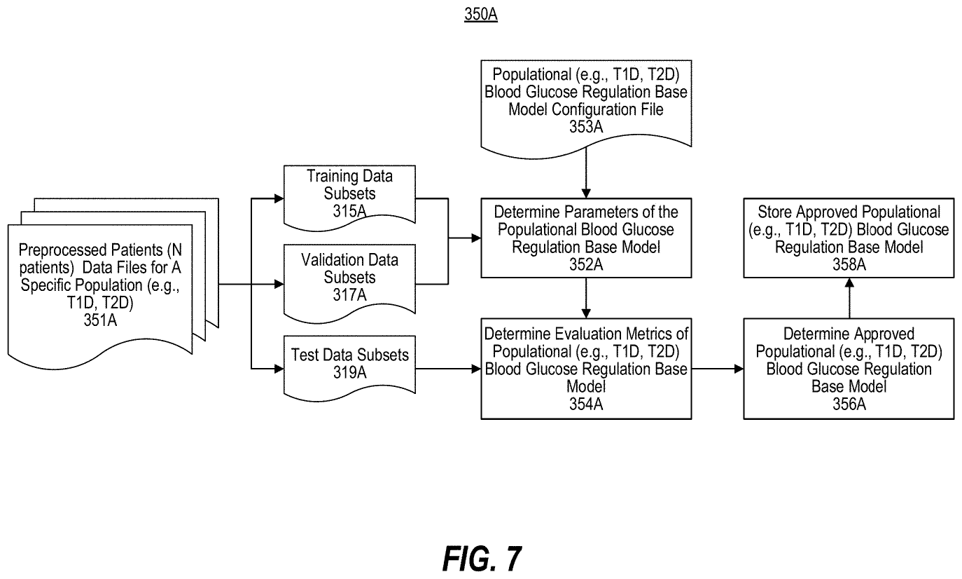

Method 10 may further include additional processes. For example, method 10 may include personalized recommendations generation process 500. In some embodiments, personalized recommendation generation process 500 may include one or more operations to provide personalized blood glucose regulation optimization strategies (e.g., bolus and/or basal insulin titration or therapy), preventive or corrective actions for an individual, and/or send signals, such as alerts or commands to insulin delivery devices, driving systems, vehicles, machines, or equipment. The operations in the processes of method 10 are described in more detail below referring to the flow charts in FIGS. 7-12.

### Patient Data Acquisition

In some embodiments, in patient data acquisition process 100, patient data are acquired from one or more devices or sources. Patient data may include one or more types of data. In some embodiments, the patient data include data points acquired at one or more times over one or more periods. As shown in FIG. 3, the patient data are received and stored in a database 111. As shown in FIG. 3, the patient data are received and stored by a database 111 to be further processed and used for generating a personalized blood glucose regulation model in process 300. As shown in FIG. 3, the data sources may include, but not limited to, glucometers 101 (e.g., continuous glucose monitors (CGM), blood glucose meters using test strips, and non-invasive blood glucose meters), insulin delivery devices 102 (e.g., insulin delivery syringes, pens, pumps, or artificial pancreas systems), activity tracking devices 103 (e.g., fitness trackers, smartwatches, or other wearable or implantable sensors), personal data recordings 104 (e.g., personal recordings of nutritional intake, insulin intake, medicine intake, physical activity in an electronic device, disease, physiological condition), mobile devices 105 (e.g., smart phones, tablets), electronic health records (EHR) 106, personal health records (PHR), or other medical recordings (e.g., health or medical data acquired at a clinic or hospital), nutritional database 107 (e.g., nutritional information of different categories or types of food), and personal behavior inputs 108 (e.g., routine regularity, recommendations adoption, alcohol intake, or sleeping data).

In some embodiments, the patient data acquired in process 100 include nutritional intake data acquired at a plurality of time points over one or more periods. The nutritional intake data may include carbohydrate intake, including one or more of the amount of carbohydrate and the type of carbohydrate taken in by the patient. In some embodiments, the nutritional intake data includes protein intake, including one or more of the amount of protein and the type of protein taken in by the patient. In some embodiments, the nutritional intake data includes fat or lipid intake, including one or more of the amount of fat or lipid and the type of fat or lipid taken in by the patient. The nutritional intake data may include other information. In some embodiments, the nutritional intake data includes fibre intake, including one or more of the amount of fibre and the type of fibre taken in by the patient. In some embodiments, the nutritional intake data includes one or more of the category of food and the type of food taken in by the patient. In some embodiments, the nutritional intake data includes calorie intake from food, drinks, dietary supplements, and/or medicine taken by the patient. In some embodiments, the nutritional intake data are obtained from one or more of personal data recordings 104, mobile devices 105, electronic health records (EHR) 106, personal health records (PHR), or other medical recordings, and nutritional database 107.

In some embodiments, the patient data acquired in process 100 include blood glucose level measurements at a plurality of time points over one or more periods. In some embodiments, the blood glucose level measurements include interstitial glucose measurements. The blood glucose level measurements may be obtained from one or more of glucometers 101, insulin delivery devices 102, activity tracking devices 103, personal data recordings 104, mobile devices 105, and electronic health records (EHR) 106, personal health records (PHR), or other medical recordings.

In some embodiments, the patient data acquired in process 100 include insulin intake data for a plurality of time points over the first period. In some embodiments, the insulin intake data includes at least an amount of a bolus insulin intake. In some embodiments, the insulin intake data includes an amount of a basal insulin intake. In some embodiments, the insulin intake data includes a type of insulin molecules of a bolus insulin intake (e.g., regular or fast-acting insulin molecules). In some embodiments, the insulin intake data includes a type of insulin molecules of a basal insulin intake. In some embodiments, the insulin intake data includes the insulin delivery method used by the patient, such subcutaneous insulin injection (e.g. by a syringe or an insulin pen), subcutaneous insulin infusion (e.g. by a pump), intravenous insulin delivery (e.g. by a syringe or a pump), nasal insulin delivery, oral insulin delivery, colonic insulin delivery, or trans-dermal delivery. The insulin intake data may be obtained from one or more of insulin delivery devices 102, personal data recordings 104, mobile devices 105, and electronic health records (EHR) 106, personal health records (PHR), or other medical recordings.

In some embodiments, the patient data acquired in process 100 include physical activity data for a plurality of time points over one or more periods. In some embodiments, the physical activity data includes one or more of heart rate measurements, calories or kilocalories burned, steps, type of activity, duration of activity, Metabolic Equivalent of Task (MET), duration of sleep, and phase of sleep. The physical activity data may be obtained from one or more of activity tracking devices 103, personal data recordings 104, mobile devices 105, and electronic health records (EHR) 106, personal health records (PHR), or other medical recordings.

In some embodiments, the patient data acquired in process 100 include patient profile data. In some embodiments, the patient profile data includes one or more of sex, birth date, type of diabetes, target glucose range, weight, height, types of insulin molecules, and drug therapy. In some embodiments, the plurality of data sets includes body index data. In some embodiments, the plurality of data sets includes body index data at one or more time points over one or more periods. In some embodiments, the body index data includes one or more of weight, height, and body mass index (BMI).

In some embodiments, the patient data acquired in process 100 include date and time of data acquisition. In some embodiments, the patient data acquired in process 100 include the sources of the data.

### Patient Data Integration, Synchronization, and Compilation

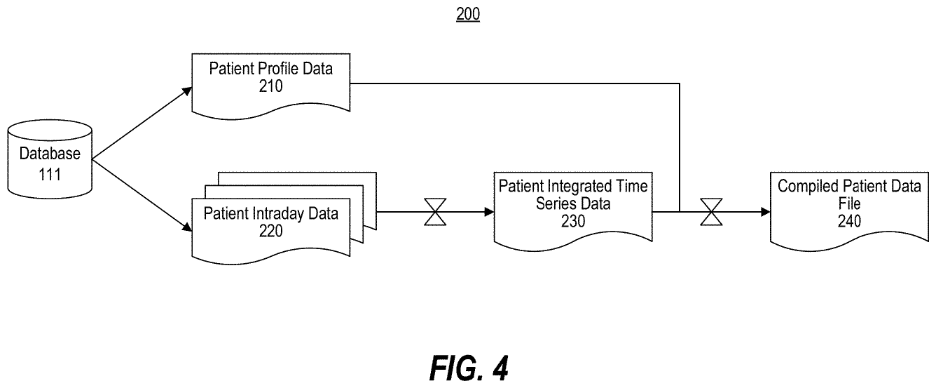

FIG. 4 is a schematic representation of an exemplary process 200 for generating compiled patient data to be used for generating a personalized blood glucose regulation model, according to some embodiments of the present disclosure. As shown in FIG. 4, in some embodiments, the patient data acquired in process 100 is collected from database 111. In some embodiments, patient profile data 210 is obtained from the patient data collected from database 111. In some embodiments, patient profile data 210 includes one or more types of profile data of a specific patient, including, but not limited to, sex, birth date, type of diabetes, target glucose range, weight, height, types of insulin molecules, and previous and/or current drug therapy.

In some embodiments, patient intraday data 220 is collected from database 111. In some embodiments, patient intraday data 220 include the patient data acquired in process 100 from multiple devices or sources over one or more periods. Patient intraday data 220 for one day and/or multiple days, such as one or more weeks or one or more months, may be collected from database 111. In some embodiments, patient intraday data 220 are integrated into time series data 230 over one or more periods within one day. In some embodiments, the integrated time series data 230 includes a plurality of data sets. In some embodiments, the plurality of data sets of the integrated time series data 230 includes one or more of the above-described nutritional intake data, insulin intake data, physical activity data, and body index data.

In some embodiments, each of the plurality of data sets includes data points indexed in time order over one or more periods. Each data point of a data set may include an indication of the date and time of data acquisition. In some embodiments, the data points of a data set are taken at successive and equally spaced points in time. In some embodiments, the data points of a data set are taken at successive but different spaced points in time. In some embodiments, the data points of different data sets are taken at the same points in time. In some embodiments, the data points of different data sets are taken at different points in time. In some embodiments, the data points of different data sets are taken at equally spaced points in time. In some embodiments, the data points of different data sets are taken at different spaced points in time. In some embodiments, the data points of different data sets are taken over the same period within one day. In some embodiments, the data points of different data sets are taken over different periods within one day.

In some embodiments, the plurality of data sets of the patient integrated time series data 230 are synchronized or aligned in time order. In some embodiments, as shown in FIG. 4, patient profile data 210 and patient integrated time series data 230 of a patient is combined and compiled into a compiled patient data file 240 to be used for generating personalized blood glucose regulation model. In some embodiments, the compiled patient data file 240 is stored in database 111.

### Personalized Blood Glucose Regulation Model Generation

The compiled patient data file 240 generated in process 200 is used for generating a personalized blood glucose regulation model in personalized blood glucose regulation model generation process 300. As shown in FIG. 3, personalized blood glucose regulation model generation process 300 may perform one or more of a plurality of processes to determine a personalized blood glucose regulation model. In some embodiments, personalized blood glucose regulation model generation process 300 may perform process 300A. In some embodiments, personalized blood glucose regulation model generation process 300 may perform process 300B. In some embodiments, personalized blood glucose regulation model generation process 300 may perform both of processes 300A and 300B. Each of processes 300A and 300B includes one or more operations as described below.

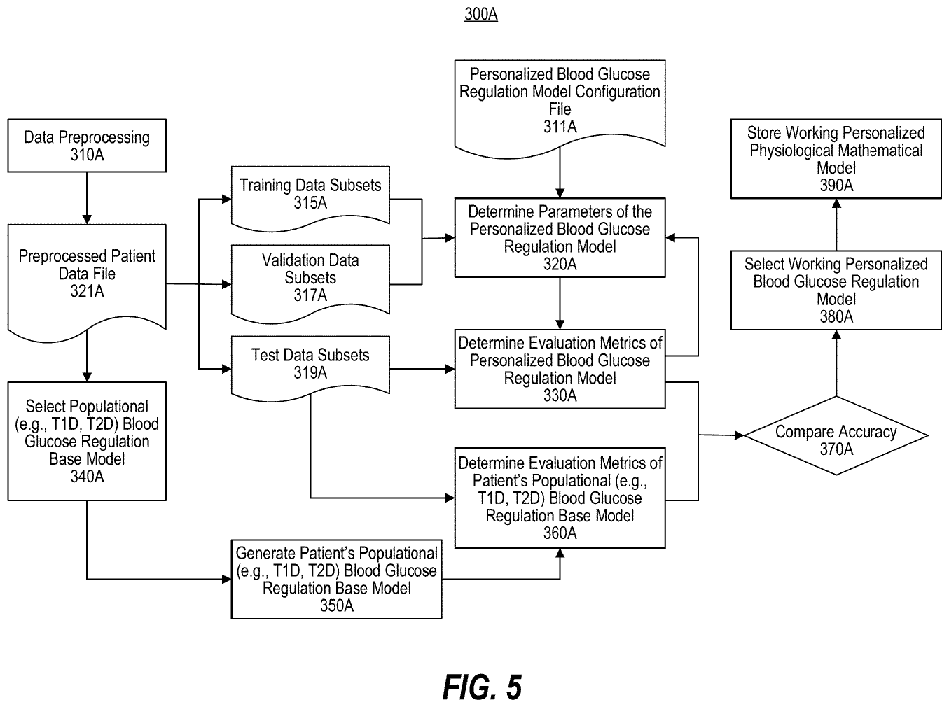

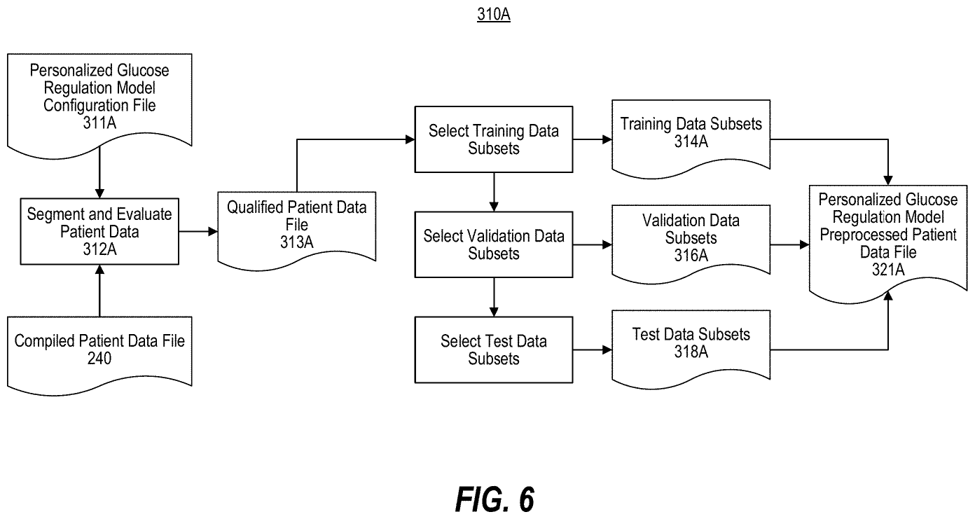

FIG. 5 is a schematic representation of an exemplary process 300A for generating a personalized blood glucose regulation model, according to some embodiments of the present disclosure. As shown in FIG. 5, in some embodiments, process 300A includes processes 310A, and 320A. Process 310A is a data preprocessing step at which the compiled patient data file 240 generated in process 200 is segmented and evaluated to be used for generating a personalized blood glucose regulation model. In some embodiments, process 310A creates a preprocessed patient data file 321A. In some embodiments, preprocessed patient data file 321A includes a training data set, a validation data set, and a test data set. In some embodiments, the training data set includes one or more training data subsets 314A. In some embodiments, the validation data set includes one or more validation data subsets 316A. In some embodiments, the test data set includes one or more test data subsets 318A. The creation of preprocessed patient data file 321A is described below referring to FIG. 6.

In process 320A, in some embodiments, a set of parameters of a personalized blood glucose regulation model for an individual based on the data and signals of the individual are acquired. In some embodiments, the parameters of the personalized blood glucose regulation model are obtained using an optimization algorithm. In some embodiments, the personalized blood glucose regulation model mimics the blood glucose regulation process of an individual. In some embodiments, the personalized blood glucose regulation model mimics the blood glucose regulation process of an individual during a period of a day. In some embodiments, the personalized blood glucose regulation model mimics the blood glucose regulation process of an individual under a physiological condition, such as pregnancy, menses, infection, etc. In some embodiments, multiple personalized blood glucose regulation models are obtained for different periods of a day or for different physiological conditions.

The personalized blood glucose regulation model may be represented in various forms. In some embodiments, the personalized blood glucose regulation model can be represented by, but not limited to, the equations below:

$$\frac{d G}{d t} = {f}_{GI} ( {c}_{M} , {c}_{D} , {c}_{P} , l , p , t ) + {f}_{E G P} ( I ( t ) ) - {f}_{G C} ( G ( t ) , hr , I ( t ) )$$

$$\frac{dI}{d t} = {f}_{II} ( m , t ) + {f}_{PIP} ( G ( t ) ) - {f}_{ID} ( I ( t ) )$$

where G(t) represents glucose mass at a function of time, t represents time, dG/dt represents the rate of glucose mass changes, I(t) represents insulin dose as a function of time, dI/dt represents the rate of insulin dose changes, c_{M} represents monosaccharide carbohydrate mass taken in at a specific time, CD represents disaccharide carbohydrate mass taken in at a specific time, c_{P} represents polysaccharide carbohydrate mass taken in at a specific time, p represents protein mass taken in at a specific time, I represents lipid mass taken in at a specific time, m represents the dose of a certain insulin molecule taken in at a specific time, hr(t) represents heart rate as a function of time, f_{GI} represents a glucose intake function, f_{EGP} represents an endogenous glucose production function, f_{GC} represents a glucose consumption function, f_{H} represents an insulin infusion function, f_{PIP} represents a pancreatic insulin production function, and f_{ID} represents an insulin degradation function. Each function in the above equations include one or more parameters to be determined in process 320A. In embodiments, an optimized set of parameters of the personalized blood glucose regulation model is determined in process 320A.

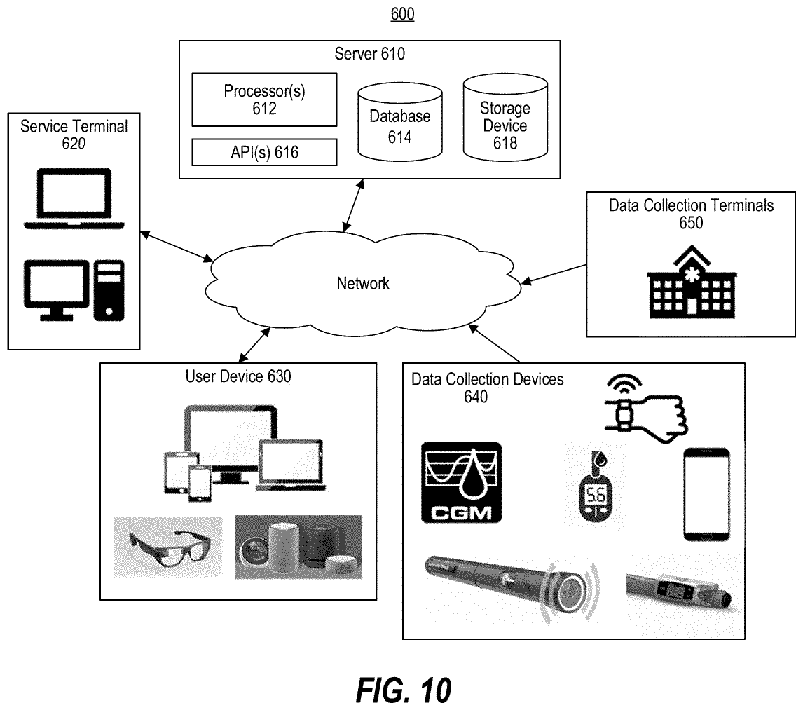

In some embodiments, process 300A includes process or step 330A. In some embodiments, step 330A includes evaluating the personalized blood glucose regulation model having a set of optimized parameters by determining one or more evaluation metrics using one or more test data subsets 319A. In some embodiments, an overall evaluation of the accuracy of the personalized blood glucose regulation model is obtained in step 330A based on the determined one or more evaluation metrics. In some embodiments, step 330A further includes determining whether the determined one or more evaluation metrics meet one or more predetermined criteria. In response to determining that the one or more evaluation metrics do not meet the one or more predetermined criteria, process 300A repeats process 310A and/or process 320A to obtain another set of optimized parameters for the personalized blood glucose regulation model. In response to determining that the one or more evaluation metrics do meet one or more predetermined criteria, the personalized blood glucose regulation model with the optimized parameters is designated as the approved personalized blood glucose regulation model. In some embodiments, the approved personalized blood glucose regulation model is saved in a storage device, such as database 111 or a non-transitory computer-readable medium of a server or a service terminal (FIG. 10). In some embodiments, the approved personalized blood glucose regulation model is used to generate blood glucose level predictions.

Processes 310A, 320A, and 330A are described in further detail in the sections below.

Data Preprocessing

FIG. 6 is a schematic representation of an exemplary process 310A for preprocessing compiled patient data file 240 for generating a personalized blood glucose regulation model, according to some embodiments of the present disclosure. In some embodiments, as shown in FIG. 6, process 310A receives compiled patient data file 240 and a personalized blood glucose regulation model configuration file 311A and creates preprocessed patient data file 321A. As shown in FIG. 6, at step 312A, compiled patient data file 240 and configuration file 311A are collected, for example, from database 111 or from a service terminal (shown in FIG. 10). In some embodiments, configuration file 311A is generated from a template. In some embodiments, configuration file 311A includes preprocessing configurations, optimization configurations, and testing configurations. Exemplary embodiments for the preprocessing configurations are described below. Exemplary embodiments for the optimization configurations and testing configurations are described further below referring to FIG. 5.

In some embodiments, the preprocessing configurations include one or more indicators for evaluating and/or qualifying the data sets in compiled patient data file 240. Examples of the indicators include, but not limited to, the length of the time interval between successive BGL measurements, lack of basal insulin intake records according to the patient's insulin therapy, lack of nutritional intake record for one or more periods during the day, inaccuracy of the source of data, and lack of heart rate records. Various indicators that provide information on the quality or reliability of the data sets in compiled patient data file 240 may be included and not limited to the examples provided herein. The types or numbers of indicators may be determined based on the types or sources of the data sets in compiled patient data file 240.

In some embodiments, the data sets in compiled patient data file 240 are evaluated based on the one or more indicators in configuration file 311A. In some embodiments, a qualified patient data file 313A is generated from the compiled patient data file 240 after the evaluation process.

In some embodiments, data sets in compiled patient data file 240 may be evaluated and qualified according to a predetermined protocol in configuration file 311A. In one exemplary protocol, in an initial step, a data set is evaluated with a full score, e.g., 100. In some embodiments, the full score is predetermined based on the total number of indicators used for evaluating the data set. In the next step, the score for the data set is penalized or reduced if one or more indicators are present in the data set. In some embodiments, data sets having a score equal to and/or above a predetermined threshold are retained in the qualified patient data file 313A. In some embodiments, data sets are ranked in accordance with their scores in qualified patient data file 313A. In some embodiments, the priority level of a data set in patient data file 313A is set in accordance with its score. In some embodiments, a data set having a higher score in patient data file 313A is given more weight or deemed more reliable when used for creating training data subsets 314A, validation data subsets 316A, or testing data subsets 318A.

As a non-limiting example, one indicator is the length of the time interval between successive BGL measurements. The score of the data set may be reduced in proportion to the length of the time interval between successive BGL measurements in the data set. A longer time interval between successive BGL measurements may cause a higher reduction of the score of the data set. The reduction may be a number ranging from 0 to 100 divided by the total number of indicators, for example.

As another non-limiting example, one indicator is the lack of basal insulin intake record according to the patient's insulin therapy in the data set. If such indicator is present in a data set, the score of the data set may be reduced by 100 divided by the total number of indicators. As another non-limiting example, one indicator is the lack of nutritional intake record for one or more periods during the day. If such indicator is present in a data set, the score of the data set may be reduced by 100 divided by the total number of indicators.

As another non-limiting example, one indicator is accuracy or reliability of the source of data. Certain sources of data may provide more accurate records than other sources of data. For example, glucometers 101, insulin delivery devices 102, and activity tracking devices 103, electronic health records (EHR) 106 may provide more reliable and accurate data than personal data recordings 104. Among the activity tracking devices 103, wearable fitness devices may provide more reliable and accurate data than mobile phones. The reliability of the sources of data may be ranked and adjusted based on the individual's usage of the data sources, the operating states of the data collection devices, or other considerations. If the source of a data set is determined to be from an unreliable source, the score of the data set may be reduced by a number ranging from 0 to 100 divided by the total number of indicators.

As another non-limiting example, one indicator is the lack of sufficient heart rate record in the data set. For example, a heart rate recording ratio, that is the duration without heart rate recording divided by a time interval (e.g., six hours), of the data set may be determined. The score of the data set may be reduced in proportion to the heart rate recording ratio by a number ranging from 0 to 100 divided by the total number of indicators.

In some embodiments, the preprocessing configurations in the configuration file 311A include one or more predetermined periods during a day for creating training data subsets 314A from the data sets in qualified patient data file 313A. Each training data subset 314A may correspond to a predetermined period during a day. The one or more periods for creating training data subsets 314A may be predetermined based on the different physiological states or different metabolic rates of an individual during a day. For example, the preprocessing configurations may include a fasting period, a resting meal period, and a physical exercise period. The fasting period may start some time, e.g., 5-6 hours, after the last evening meal, and may end with the first meal of the day. The resting meal period may start some time, e.g., 1-3 hours, before a meal that was not eaten at dawn, and may end at some time, e.g., 4-10 hours, after that meal, provided that no physical activity was performed during that period. The physical exercise period may start some time, e.g., 1-3 hours before a physical exercise, and may end at some time, e.g., 4-10 hours, after that exercise.

The start time of a first training data subset may be the start of the fasting period and the end time of the first training data subset may be the end of the fasting period. The start time of a second training data subset may be the start of the resting meal period and the end time of the second training data subset may be the end of the resting meal period. The start time of a third training data subset may be the start of the physical exercise period and the end time of the third training data subset may be the end of the physical exercise period.

In some embodiments, the preprocessing configurations in configuration file 311A includes a predetermined value for the number of data sets in a training data subset 314A. In some embodiments, the predetermined number of data sets in a training data subset 314A are selected from the data sets in qualified patient data file 313A. In some embodiments, a random number of data sets in a training data subset 314A are selected from the data sets in qualified patient data file 313A.

In some embodiments, the preprocessing configurations in the configuration file 311A include various configurations for creating validation data subsets 316A similar to those described above for creating training data subsets 314A. In some embodiments, the preprocessing configurations in the configuration file 311A may include one or more predetermined periods during a day for creating validation data subsets 316A. The one or more predetermined periods may include a fasting period, a resting meal period, and a physical exercise period. Each validation data subset 316A may correspond to a predetermined period of a day.

In some embodiments, the preprocessing configurations in configuration file 311A includes a predetermined value for the number of data sets in a validation data subsets 316A. In some embodiments, the predetermined number of data sets in a validation data subset 316A are selected from the data sets in qualified patient data file 313A. In some embodiments, a random number of data sets in a validation data subset 316A are selected from the data sets in qualified patient data file 313A.

As described herein, the data sets in validation data subsets 316A differ from the data sets in the training data subsets 314A. In some embodiments, as shown in FIG. 6, the data sets in validation data subset 316A are selected after the data sets in training data subsets 314A are selected. The data sets in validation data subset 316A are selected from the data sets in qualified patient data file 313A without the data sets that have been selected for training data subsets 314A.

In some embodiments, the preprocessing configurations in the configuration file 311A include various configurations for creating test data subsets 318A. In some embodiments, the preprocessing configurations in configuration file 311A includes a predetermined value for the number of data sets in a test data subset 318A. In some embodiments, the preprocessing configurations in the configuration file 311A include one or more predetermined periods for creating test data subsets 318A from the data sets in qualified patient data file 313A. Each test data subset 318A may correspond to a predetermined period during a day. The one or more periods for creating test data subsets 318A may be predetermined based on a schedule of food intake or nutritional intake during a day. For example, the preprocessing configurations may include a dawn period (e.g., from 00:00 to 06:00 in a 24-hour period), a morning period (e.g., from 06:00 to 12:00 in a 24-hour period), an afternoon period (e.g., 12:00 to 18:00 in a 24-hour period), a night period (e.g., from 18:00 to 24:00 in a 24-hour-period), and an all-day period. In some embodiments, the dawn period, the morning period, the afternoon period, and the night periods are consecutive periods of a day. In some embodiments, the all-day period is the combination of the dawn period, the morning period, the afternoon period, and the night periods of a day in time order.

As described herein, the data sets in test data subsets 318A differ from the data sets in the validation data subset 316A and different from the data sets in the training data subsets 314A. In some embodiments, as shown in FIG. 6, the data sets in test data subsets 318A are selected after the data sets in training data subsets 314A and validation data subset 316A are selected. The data sets in test data subsets 318A are selected from the data sets in qualified patient data file 313A without the data sets that have been selected for training data subsets 314A and for validation data subset 316A. In some embodiments, the period of a test data subset 318A does not overlap with the period of any of the training data subsets 314A.

In some embodiments, a personalized blood glucose regulation model preprocessed patient data file 321A is generated. In some embodiments, preprocessed patient data file 321A includes training data subsets 314A and testing data subsets 318A. In some embodiments, preprocessed patient data file 321A includes training data subsets 314A, validation data subsets 316A, and testing data subsets 318A. In some embodiments, preprocessed patient data file 321A includes the preprocessing configurations in configuration file 311A. In some embodiments, preprocessed patient data file 321A includes the optimization configurations in configuration file 311A. In some embodiments, preprocessed patient data file 321A includes the testing configurations in configuration file 311A.

Personalized Blood Glucose Regulation Model Training

As shown in FIG. 5, preprocessed patient data file 321A is used as input to process 300A generate a personalized blood glucose regulation model, according to some embodiments of the present disclosure. In process 320A, in some embodiments, training data subsets 314A are used to train or determine a set of parameters of a personalized blood glucose regulation model for an individual using an optimization algorithm. The optimization algorithm may be any suitable algorithm that allow for optimizing the parameters of the personalized blood glucose regulation model.

Non-limiting examples of the optimization algorithm may include stochastic algorithms (e.g., genetic algorithms or particle swarm algorithms), deterministic algorithms (e.g., gradient descent algorithms), and machine learning algorithms. Machine learning algorithms may include regression algorithms, instance-based algorithms, regularization algorithms, decision tree algorithms, Bayesian algorithms, clustering algorithms, association rule learning algorithms, artificial neural network algorithms, and deep learning algorithms. Non-limiting examples of deep learning algorithms include, but not limited to, random forest algorithms, convolutional neural networks (CNNs), long short term memory networks (LSTMs), recurrent neural networks (RNNs), generative adversarial networks (GANs), radial basis function networks (RBFNs), multilayer perceptrons (MLPs), self-organizing maps (SOMs), deep belief networks (DBNs), and restricted Boltzmann machines (RBMs).

In some embodiments, the optimization configurations in the configuration file 311A include a selection of an optimization algorithm. In some embodiments, the optimization configurations in the configuration file 311A include values of one or more model parameters for controlling the optimization algorithm for training or optimizing the set of parameters of a personalized blood glucose regulation model in process 320A. Non-limiting examples of such parameters may include hyperparameters for a machine learning algorithm, hyperparameters for a genetic optimization algorithm, ranges of the chromosome gene values of a genetic algorithm, and quantity of each training data subset. The values and types of the model parameters may be determined based on the type of optimization algorithm selected for generating the personalized blood glucose regulation model. In some embodiments, validation data subsets 316A are used to evaluate a model fit on the training data subsets 314A while tuning the model parameters (e.g., hyperparameters). In some embodiments, for certain optimization algorithms, validation data subsets 316A are not used. In such instances, validation data subsets 316A may not be created in process 310A.

In some embodiments, the optimization configurations include at least one stopping criterion for ending the optimization algorithm in process 320A. Non-limiting examples of a stopping criterion may be the number of iterations, time, the value of a fitness function, a random termination key, and the accuracy of the generated personalized blood glucose regulation model (e.g., one or more evaluation metrics of the generated personalized blood glucose regulation model).

In some embodiments, training or optimization of the set of parameters of a personalized blood glucose regulation model in process 320A are performed in multiple steps based on the number of training data subsets 314A. For example, training data subsets 314A may include three data subsets, a first training data subset for the fasting period, a second training data subset for the resting meal period, and a third training data subset for the physical exercise period. In some embodiments, correspondingly, validation data subsets 316A may include three data subsets, a first validation data subset for the fasting period, a second validation data subset for the resting meal period, and a third validation data subset for the physical exercise period. In a first step, in some embodiments, training or optimization of a personalized blood glucose regulation model using an optimization algorithm may be performed using only the first training data subset or the first training and validation data subsets. In the first step, a first subset of the parameters of the personalized blood glucose regulation model relevant for the fasting period can be obtained or updated. Similarly, in a second step, in some embodiments, training or optimization of a personalized blood glucose regulation model using an optimization algorithm may be performed using only the second training data subset or the second training and validation data subsets. In the second step, a second subset of parameters of the personalized blood glucose regulation model relevant for the resting meal period can be obtained or updated. Further, in a third step, in some embodiments, training or optimization of a personalized blood glucose regulation model using an optimization algorithm may be performed using only the third training data subset or the third training and validation data subsets. In the third step, a third subset of parameters of the personalized blood glucose regulation model relevant for the physical exercise period can be obtained. In some embodiments, the multiple steps are repeatedly performed in a number of iterations until all the subsets of parameters are optimized or at least one stopping criterion is met.

Personalized Blood Glucose Regulation Model Testing

In some embodiments, the testing configurations in configuration file 311A include criteria or thresholds values for one or more evaluation metrics for evaluating the accuracy of the optimized personalized blood glucose regulation model determined in process 320A. As a non-limiting example, one evaluation metric is the difference between measured BGL and BGL calculated using the optimized personalized blood glucose regulation model based on the test data subsets. As another non-limiting example, one evaluation metric is the difference between measured BGL and BGL calculated using the optimized personalized blood glucose regulation model and only basal insulin intake data sets over a full day based on the test data subsets. As another non-limiting example, one evaluation metric is the difference between an insulin sensitivity factor calculated using a standard accepted insulin therapy protocol and the insulin sensitivity factor calculated using the optimized personalized blood glucose regulation model based on the test data subset. Insulin Sensitivity Factor (ISF) or correction factor typically determines how much one unit of rapid or regular insulin will lower blood glucose. As another non-limiting example, one evaluation metric is the difference between an insulin-to-carb ratio calculated using a standard accepted insulin therapy protocol and the insulin-to-carb ratio calculated using the optimized personalized blood glucose regulation model based on the test data subsets. In some embodiments, the difference between measured BGL and calculated BGL for a particular evaluation metric is determined using a statistical measure, such as Mean Absolute Percentage Error (MAPE), Root Mean Square Error (RMSE), or R-squared. In some embodiments, the difference between measured BGL and calculated BGL for a particular evaluation metric is determined using a standard clinical analytical tool, such as Clarke Error Grid or Consensus Error Grid.

In some embodiments, in step 330A, one or more evaluation metrics of the optimized personalized blood glucose regulation model determined in process 320A are obtained. In some embodiments, the determined one or more evaluation metrics are compared with the criteria or threshold values in the testing configurations. In some embodiments, in response to determining that the one or more evaluation metrics meet the criteria or threshold values, the optimized personalized blood glucose regulation model is given an overall evaluation status of “approved.” The “approved” personalized blood glucose regulation model may then be saved in a storage device, such as database 111 or a non-transitory computer-readable medium of a server or a service terminal (FIG. 10). In some embodiments, in response to determining that the one or more evaluation metrics do not meet the criteria or threshold values, the optimized personalized blood glucose regulation model is given an overall evaluation status of “disapproved.” In such instances, processes 310A and/or process 320A may be repeated to obtain another optimized personalized blood glucose regulation model.

In some embodiments, multiple personalized blood glucose regulation models are obtained. The multiple personalized blood glucose regulation models may have different set of parameters. For example, multiple personalized blood glucose regulation models can be obtained using different training data subsets 314A, different validation data subsets 316A, different stopping criteria, or different optimization algorithms. In some embodiments, at least one evaluation metric of each of the multiple personalized blood glucose regulation models is determined using same test data subsets 318A. In some embodiments, the values of the evaluation metrics of the multiple personalized blood glucose regulation models are compared. In some embodiments, the multiple personalized blood glucose regulation models are ranked based on the values of the evaluation metrics. In some embodiments, the personalized blood glucose regulation model that has the best accuracy among the multiple personalized blood glucose regulation models is given the status of “approved.” In some embodiments, the personalized blood glucose regulation model that has the highest accuracy among the multiple personalized blood glucose regulation models is designated as the approved personalized blood glucose regulation model.

### Populational Blood Glucose Regulation Base Model Generation

In some embodiments, process 300A includes steps for generating a populational blood glucose regulation base model and compares the accuracy of the personalized blood glucose regulation model determined in process 320A with the accuracy of the populational blood glucose regulation base model to determine a working personalized blood glucose regulation model that has better accuracy. As shown in FIG. 5, in some embodiments, process 300A further include processes 340A, 350A, 360A, 370A, 380A, and 390A.

As described herein, the populational blood glucose regulation base model is a predetermined glucose regulation model for a patient population to which an individual belongs. In some embodiments, the populational blood glucose regulation base model can be represented by one or more equations. In some embodiments, the populational blood glucose regulation base model can be represented by the same equations described above for the personalized blood glucose regulation model. In process 340A, a populational blood glucose regulation base model including a set of parameters and functions is selected based on one or more characteristics of the patient population. In some embodiments, the one or more characteristics include one or more patient demographics, type of diabetes, level of physical activity, type of insulin molecules taken, type of diet, target glucose range, weight, height, types of insulin molecules, disease, drug therapy, or one or more of body index data (e.g., weight, height, or body mass index (BMI)).

In process 350A, a populational blood glucose regulation base model is generated. FIG. 7 is a schematic representation of an exemplary process for generating a populational blood glucose regulation base model, according to some embodiments of the present disclosure. In some embodiments, process 350A includes process 310A that creates a preprocessed patient data file for a patient population. In some embodiments, preprocessed patient data file 351A are created from compiled patient data files 240 generated in process 200 for a number of patients belong to a specific patient population. In some embodiments, to create preprocessed patient data file 351A, process 310A receives compiled patient data files 240 of the number of patients and a populational blood glucose regulation base model configuration file 353A and creates preprocessed patient data file 351A.

In some embodiments, the compiled patient data files 240 and configuration file 353A may be collected, for example, from database 111 or from a service terminal (shown in FIG. 10). In some embodiments, configuration file 353A is generated from a template. In some embodiments, configuration file 353A includes preprocessing configurations, optimization configurations, and testing configurations. In some embodiments, the preprocessing configurations in configuration file 353A include one or more of the preprocessing configurations in configuration file 311A described above. In some embodiments, preprocessing configurations in configuration file 353A further include one or more of the number of patients from whom data are collected, one or more characteristics of the patient population, the number of patients in training data subsets 315A, the number of patients in validation data subsets 317A, and the number of patients in test data subsets 319A.

Process 310 may use the preprocessing configurations in configuration file 353A to create training data subsets 315A, validation data subsets 317A, and test data subsets 319A similar to the way for creating training data subsets 314A, validation data subsets 316A, and test data subsets 318A. In some embodiments, process 310A generates preprocessed patient data file 351A that includes one or more training data subsets 315A, one or more validation data subsets 317A, and one or more test data subsets 319A. As described herein, training data subsets 315A, validation data subsets 317A, and test data subsets 319A include data of a number of patients belonging to the same patient population. Each training data subset 315A, validation data subset 317A, or test data subset 319A may correspond to a predetermined period during a day. The period may be determined based on the preprocessing configurations in configuration file 353A similar to the way the period for training data subset 314A, validation data subset 316A, test data subset 318A are determined.

As shown in FIG. 7, in some embodiments, process 350A includes processes 352A and 354A. In process 352A, in some embodiments, training data subsets 315A and validation data subsets 317A are used to train or determine a set of parameters of a populational blood glucose regulation base model for a patient population using an optimization algorithm. The optimization algorithm may be any suitable algorithm that allows for optimizing the parameters of the populational blood glucose regulation base model. Non-limiting examples may include the exemplary optimization algorithms for generating the personalized blood glucose regulation model described above.

In some embodiments, the optimization configurations in configuration file 353A include one or more of the optimization configurations in configuration file 311A described above. For example, the optimization configurations in the configuration file 353A may include values of one or more model parameters for controlling the optimization algorithm for training or optimizing the set of parameters of a populational blood glucose regulation base model in process 352A. The optimization configurations in the configuration file 353A may include at least one stopping criterion for ending the optimization algorithm in process 352A.

In some embodiments, training or optimization of the set of parameters of a populational blood glucose regulation base model in process 352A are performed in multiple steps based on the number of training data subsets 315A similar to the multiple steps of process 320A described above.

Populational Blood Glucose Regulation Base Model Testing

In some embodiments, the testing configurations in configuration file 353A include one or more of the testing configurations in configuration file 311A described above, such as criteria or thresholds values for one or more evaluation metrics for evaluating the accuracy of the optimized populational blood glucose regulation base model determined in process 352A. Non-limiting examples of evaluation metrics are described above referring to the testing configurations in configuration file 311A.

In some embodiments, in process 330A, one or more evaluation metrics of the populational blood glucose regulation base model determined in process 352A are obtained. In some embodiments, the determined one or more evaluation metrics are compared with the criteria or threshold values in the testing configurations. In some embodiments, in step 356A, in response to determining that the one or more evaluation metrics meet the criteria or threshold values, the optimized populational blood glucose regulation base model is given an overall evaluation status of “approved.” In step 358A, the “approved” populational blood glucose regulation base model may then be saved in a storage device, such as database 111 or a non-transitory computer-readable medium of a server or a service terminal (FIG. 10). In some embodiments, in response to determining that the one or more evaluation metrics do not meet the criteria or threshold values, the optimized populational blood glucose regulation base model is given an overall evaluation status of “disapproved.” In such instances, processes 310A and/or process 352A may be repeated to obtain another optimized populational blood glucose regulation base model.

In some embodiments, multiple populational blood glucose regulation base models are obtained. The multiple populational blood glucose regulation base model may have different set of parameters. For example, multiple populational blood glucose regulation base model can be obtained using different training data subsets 315A, different validation data subsets 317A, different stopping criteria, or different optimization algorithms. In some embodiments, at least one evaluation metric of each of the multiple populational blood glucose regulation base model is determined using same test data subsets 319A. In some embodiments, the values of the evaluation metrics of the multiple populational blood glucose regulation base model are compared. In some embodiments, the multiple populational blood glucose regulation base models are ranked based on the values of the evaluation metrics. In some embodiments, the populational blood glucose regulation base model that has the best accuracy is given the status of “approved.” In some embodiments, the populational blood glucose regulation base model that has the highest accuracy is determined as the approved populational blood glucose regulation base model in step 356A.

Working Personalized Blood Glucose Regulation Model Selection

In some embodiments, as shown in FIG. 5, process 300A further includes steps 360A, 370A, 380A, and 390A. In step 360A, the populational blood glucose regulation base model generated by process 350A, e.g., the approved populational blood glucose regulation base model, is retrieved and evaluated using same test data subsets 318A as used for evaluating the personalized blood glucose regulation model in step 330A. In some embodiments, the same evaluation metrics are obtained in step 360A as in step 330A. In some embodiments, in step 370A, the values of the evaluation metrics of the populational blood glucose regulation base model and the personalized blood glucose regulation models are compared. In some embodiments, in step 380A, the model that has the higher accuracy is selected as the working personalized blood glucose regulation model. In some embodiments, in step 390A, the working personalized blood glucose regulation model is saved in a storage device, such as database 111 or a non-transitory computer-readable medium of a server or a service terminal (FIG. 10). The working personalized blood glucose regulation model may be used for generating blood glucose level predictions as described below.

### Hybrid Personalized Blood Glucose Model Generation

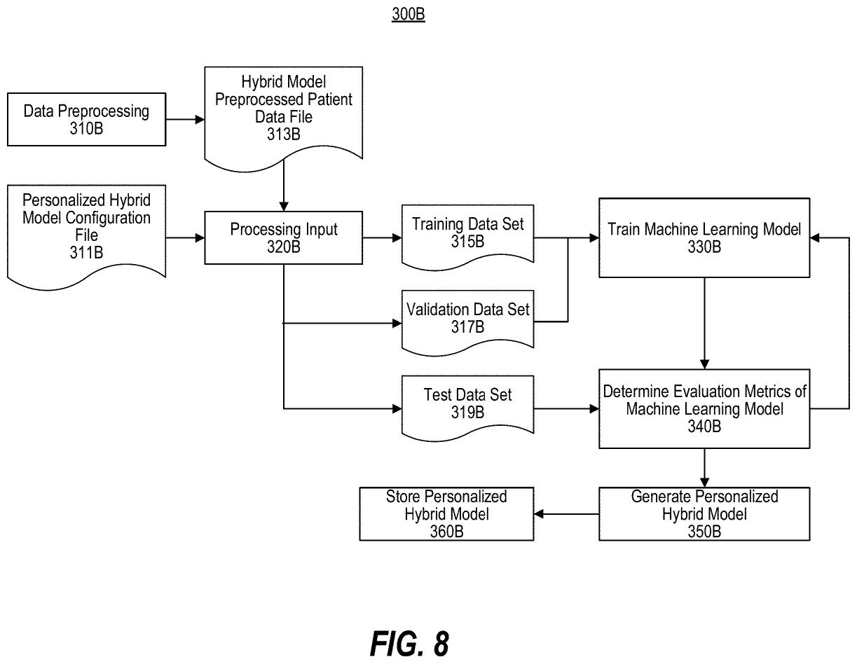

In some embodiments, as shown in FIG. 3, personalized blood glucose regulation model generation process 300 may perform process 300B to generate a hybrid personalized blood glucose regulation model. FIG. 8 is a schematic representation of an exemplary process for generating a hybrid personalized blood glucose regulation model, according to some embodiments of the present disclosure.

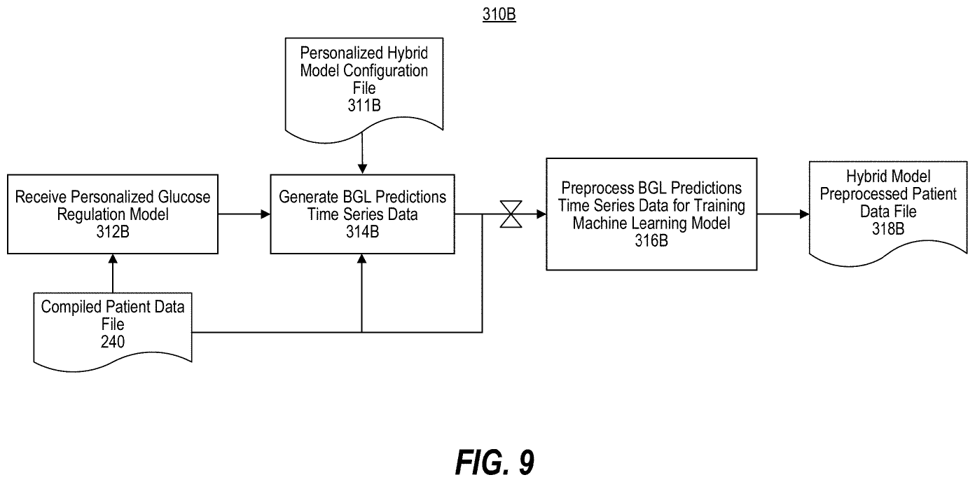

As shown in FIG. 8, in some embodiments, process 300B includes processes 310B, 320B, 330B, and 340B. Process 310B is a data preprocessing step at which the compiled patient data file 240 generated in process 200 and an optimized personalized blood glucose regulation model generated in process 300A are used to create a hybrid model preprocessed patient data file 313B. The creation of preprocessed patient data file 313B is described below referring to FIG. 9. Process 320B receives preprocessed patient data file 313B and a personalized hybrid model configuration file 311B. In process 320B, data sets in preprocessed patient data file 313B are used to create a training data set 315B, a validation data set 317B, a test data set 319B in accordance with configurations in personalized hybrid model configuration file 311B. In process 330B, a machine learning model is trained using training data set 315B and validation data set 317B. In process 340B, one or more evaluation metrics of the trained machine learning model are determined. Processes 320B, 330B, and 340B are described further below.

Data Preprocessing

FIG. 9 is a schematic representation of an exemplary process 310B for preprocessing compiled patient data file 240 for generating a hybrid personalized blood glucose regulation model, according to some embodiments of the present disclosure. In some embodiments, process 310B includes steps 312B, 314B, and 316B. As shown in FIG. 9, in some embodiments, in step 312B, a personalized blood glucose regulation model is received. In some embodiments, the personalized blood glucose regulation model is received from a storage device, such as database 111 or a non-transitory computer-readable medium of a server or a service terminal (FIG. 10).

In some embodiments, the personalized blood glucose regulation model is generated by process 310A as described above. In some embodiments, the personalized blood glucose regulation model is an optimized personalized blood glucose regulation model generated in process 320A. In some embodiments, the personalized blood glucose regulation model is an approved personalized blood glucose regulation model determined in process 330A. In some embodiments, the personalized blood glucose regulation model is an optimized populational blood glucose regulation base model generated in process 350A. In some embodiments, the personalized blood glucose regulation model is an approved populational blood glucose regulation base model determined in process 360A. In some embodiments, the personalized blood glucose regulation model is the working personalized glucose regulation base model determined in steps 380A.

In step 314B, the personalized blood glucose regulation model, compiled patient data file 240, and personalized hybrid model configuration file 311B are used to generate predicted BGL data over a prediction time interval. Compiled patient data file 240 and configuration file 311B are collected, for example, from database 111 or from a service terminal (shown in FIG. 10). In some embodiments, configuration file 311B is generated from a template.

In some embodiments, the predicted BGL data include one or more time series data sets. Each predicted data point in a time series data set includes a predicted BGL indexed with a date and time in the prediction time interval. In some embodiments, the prediction time interval is predetermined in configuration file 311B. In some embodiments, configuration file 311B further includes an input-data time interval that precedes the prediction time interval. As described above, compiled patient data file 240 includes patient integrated time series data 230 over a period and each data point of a data set in patient integrated time series data 230 includes an indication of the date and time of data acquisition. In some embodiments, data points in patient integrated time series data 230 during the input-data time interval are used as input to the personalized blood glucose regulation model to generate the predicted BGL over the prediction time interval.

In some embodiments, the input-data time interval is longer than or equal to the prediction time interval. In some embodiments, the input-data time interval is shorter or equal to the prediction time interval. In some embodiments, the input-data time interval and the prediction time interval are consecutive time intervals of a certain period. In some embodiments, the input-data time interval ranges from 0 hours to 48 hours. In some embodiments, the prediction time interval ranges from 15 minutes to 24 hours.

In some embodiments, in step 316B, patient integrated time series data 230 from compiled patient data file 240 and predicted BGL time series data are combined to generate the hybrid model time series data for training a machine learning model. More specifically, patient integrated time series data 230 in the prediction time interval are aligned in time with predicted BGL time series data generated for the prediction interval. For example, a data set containing measured BGL of patient integrated time series data 230 in the prediction time interval is aligned in time with a data set containing predicted BGL such that the measured BGL and the predicted BGL form data pairs for the prediction interval in time order. In some embodiments, the combined time series data containing the data pairs form hybrid model time series data for the prediction interval. The number of data sets in the hybrid model time series data may be equal to the number of data sets in patient integrated time series data 230 or the number of data sets in predicted BGL time series data.

In some embodiments, as shown in FIG. 9, the hybrid model time series data for the prediction time interval are further preprocessed in step 316B before being used for training a machine learning model. In some embodiments, in step 316B, the hybrid model time series data is further preprocessed using one or more data preprocessing methods to improve the quality of the data before being used to train a machine learning model. Non-limiting examples of the preprocessing methods include data filtering, data normalization, data qualification, data encoding, data standardization, data cleaning, data sampling, etc.

In some embodiments, in step 318B, a hybrid model preprocessed patient data file 313B containing the preprocessed hybrid model time series data is created. In some embodiments, the hybrid model preprocessed patient data file 313B is saved in a storage device, such as database 111 or a non-transitory computer-readable medium of a server or a service terminal (FIG. 10). Hybrid model preprocessed patient data file 313B is used for training a machine learning model as described below.

Machine Learning Model Training and Testing

As shown in FIG. 8, in some embodiments, hybrid model preprocessed patient data file 313B and personalized hybrid model configuration file 311B are received. In some embodiments, personalized hybrid model configuration file 311B includes one or more configurations of the machine learning model. Non-limiting examples of the configurations of the machine learning model include hyperparameters values, e.g., number of layers, hidden units, dropout value, activation function, training hyperparameters, e.g., learning rate, number of epochs, batch size, and data hyperparameters, e.g., train-validation-test split ratio. In some embodiments, personalized hybrid model configuration file 311B defines one or more input features of the machine learning model. Non-limiting examples of the features include measured BGL, name of food, category of food, type of food, macronutrient intake type, e.g., carbohydrate, protein, lipids, fibers, macronutrient intake quantity, macronutrient intake rate, predicted BGL, type of exercise, name of exercise, duration of exercise, type of insulin molecule, insulin intake quantity, insulin intake rate, heart rate, date, week, day, and time. In some embodiments, personalized hybrid model configuration file 311B defines one or more labels of the machine learning model, such as measured BGL, dose of basal insulin, or dose of bolus insulin.

In some embodiments, in process 320B, the hybrid model time series data from preprocessed patient data file 313B are processed into the input features defined in the hybrid model configuration file 311B. In some embodiments, in process 320B, measured BGL is determined as the label of the machine learning model. In some embodiments, in process 320B, the date sets in the hybrid model time series data are then split into training data set 315B, validation data set 317B, test data set 319B. In some embodiments, hybrid model configuration file 311B includes the protocol for splitting these data sets. The protocol may be predetermined based on the number of data sets in hybrid model time series data and the type of the machine learning model (e.g., number of hyperparameters of the machine learning model). For example, hybrid model configuration file 311B may define dataset split ratio.

In some embodiments, hybrid model configuration file 311B may define the machine learning model to be trained. As described herein, the machine learning model can be any machine learning model suitable for making predictions based on time series data. Non-limiting examples of the machine learning model include artificial neural network, neural networks, deep neural networks, deep belief networks, recurrent neural networks, transformers, convolutional neural networks, Bayesian network, linear regression models, non-linear regression models, and genetic algorithms.

In some embodiments, in step 330B, training data set 315B is used to train the machine learning model. The machine learning model is trained to predict BGL at one or more time points in a prediction time interval using input data sets over an input-data time interval. In some embodiments, the input-data time interval is before a set date and time and the prediction time interval is after the set date and time. More specifically, the machine learning model learns from the input features from the data pairs in the hybrid model time series data, whether the BGL predictions made by the personalized glucose regulation model needs to be corrected and how much correction is needed to improve the accuracy of predicted BGL.

In some embodiments, in step 330B, validation data set 317B is used to evaluate a model fit on the training data set 315B, tune the hyperparameters of the model, or implement early stopping. In some embodiments, in step 340B, test data set 319B is used for evaluating the trained machine learning model. The trained machine learning model may be evaluated based on one or more evaluation metrics. Non-limiting examples of evaluation metrics are described above. In some embodiments, the one or more evaluation metrics are defined in hybrid model configuration file 311B.

In some embodiments, step 340B further includes determining whether the determined one or more evaluation metrics of the trained machine learning model meet one or more predetermined criteria. In some embodiments, the one or more predetermined criteria are defined in hybrid model configuration file 311B. In some embodiments, in response to determining that the one or more evaluation metrics do not meet the one or more predetermined criteria, process 300B repeats process 310B, 320B, and/or process 330B to obtain another machine learning model. In response to determining that the one or more evaluation metrics do meet one or more predetermined criteria, the trained machine learning model is saved in a storage device, such as database 111 or a non-transitory computer-readable medium of a server or a service terminal (FIG. 10).

In some embodiments, in step 350B, the trained machine learning model is combined with the personalized blood glucose regulation model to generate a personalized hybrid model. For example, an optimized personalized blood glucose regulation model may be used first to predict BGL at one or more time points, and the predicted BGL are then used as input to the trained machine learning model to generate corrected predicted BGL with higher accuracy. In some embodiments, step 350B, the personalized hybrid model is determined as a working personalized blood glucose regulation model for generating blood glucose predictions. In some embodiments, in step 360B, the personalized hybrid model is saved in a storage device, such as database 111 or a non-transitory computer-readable medium of a server or a service terminal (FIG. 10).

### Exemplary Systems and Devices

Various systems and devices may be used to implement the processes of method 10 shown in FIG. 3. FIG. 10 is a schematic representation of an exemplary system 600 for implementing method 10, according to some embodiments of the present disclosure. As shown in FIG. 10, an exemplary system 600 includes a server 610, a service terminal 620, and a user device 630. In some embodiments, system 600 includes one or more collection devices 640. In some embodiments, system 600 includes one or more data collection terminals 650. In some embodiments, the server, devices, and terminals are operatively connected via one or more public or private network connections including a wireless communication network, the Internet, an intranet, a Wide-Area Network (WAN), a Metropolitan-Area Network (MAN), a Local Area Network (LAN), (e.g., a LAN compliant with the IEEE 802.11 Standards), a personal area network (PAN) (e.g., Bluetooth compliant with the IEEE 802.15 Standards), a wired communication network, or the like.

In some embodiments, as shown in FIG. 10, server 610 includes a processor 612, a database 614, and a storage device 618. The storage device 618 may include at least one non-transitory computer-readable medium containing instructions. When executed by the at least one processor, the instructions can cause processor 612 to perform one or more processes of method 10. In some embodiments, the server is provided by a cloud platform. Non-limiting example of a suitable cloud platform include Amazon Web Services (AWS), Google Cloud Platform, Microsoft Azure Cloud Services. In some embodiments, server 610 includes an application programming interface (API) 616. In some embodiments, the instructions stored in the non-transitory computer-readable medium include operations performed by the API to interface with other devices and terminals in system 600.

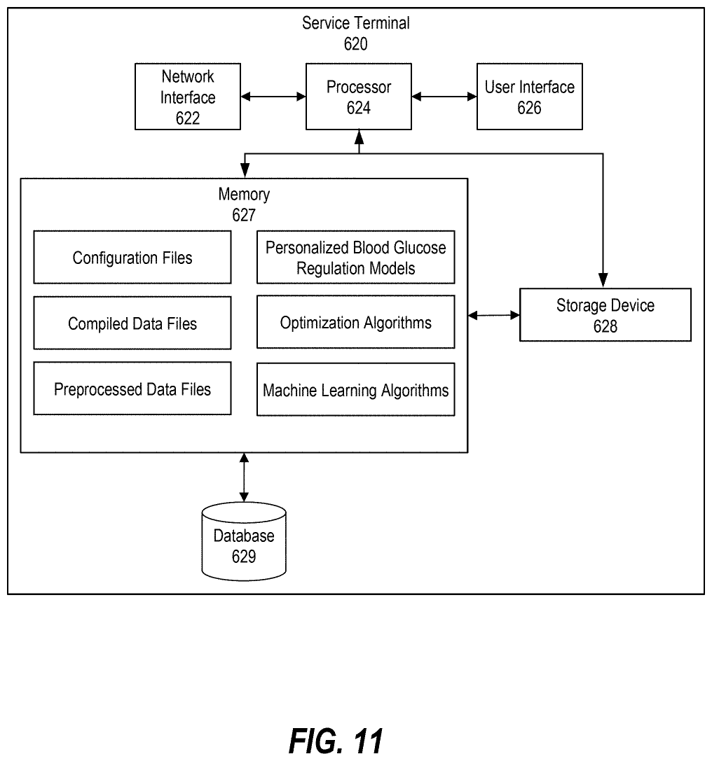

In some embodiments, service terminal 620 is a computing device having a user interface 626. Non-limiting examples of service terminal 620 include a server, a computing device, a network device, a computer, or a virtual machine. FIG. 11 is a schematic representation of an exemplary service terminal 620 for implementing method 10, according to some embodiments of the present disclosure. As shown in FIG. 11, in some embodiments, service terminal 620 includes a network interface 622 and a processor 624. In some embodiments, service terminal 620 includes one or more storage devices, such as a memory 627 and a physical storage device 628 (e.g., hard drive, solid-state drive, etc.). Memory 627 may be a random-access memory (RAM) or a read-only memory (ROM).

In some embodiments, service terminal 620 is used to generate or update one or more of configuration files 311A, 353A, and 313B. In some embodiments, one or more of configuration files 311A, 353A, and 313B are generated or updated based on one or more templates stored in storage device 618. In some embodiments, input from a user is received at user interface 626 to generate or update one or more of configuration files 311A, 353A, and 313B. In some embodiments, memory 627 stores a set of instructions for instructing processor 624 to perform at least a part of the steps or processes of method 10. In some embodiments, memory 627 and/or storage device 628 store at least a part of the data files, configuration files, models, or algorithms generated or used by method 10.

In some embodiments, user device 630 is a computing device having a user interface. Non-limiting examples of user device 630 include a mobile device (e.g., a smart phone, a tablet, or the like), a smart device (e.g., a smart wearable device, a smart speaker, a smart display, or the like), an insulin delivery device (e.g., a smart insulin pen or pump), or other like electronic device that can collect data and present information to an individual via a user interface. In some embodiments, user device 630 is used to collect data from an individual and/or provide blood glucose regulation information to the individual. The data collected from an individual may include one or more of the nutritional intake data, blood glucose level measurements, insulin intake data, physical activity data, patient profile data, body index data, or demographics data described above. The blood glucose regulation information may include prediction and/or simulation of simulate blood glucose levels, prediction or identification of risks of blood glucose excursions, personalized recommendations for blood glucose regulation, and/or personalized recommendations for preventive or corrective actions.

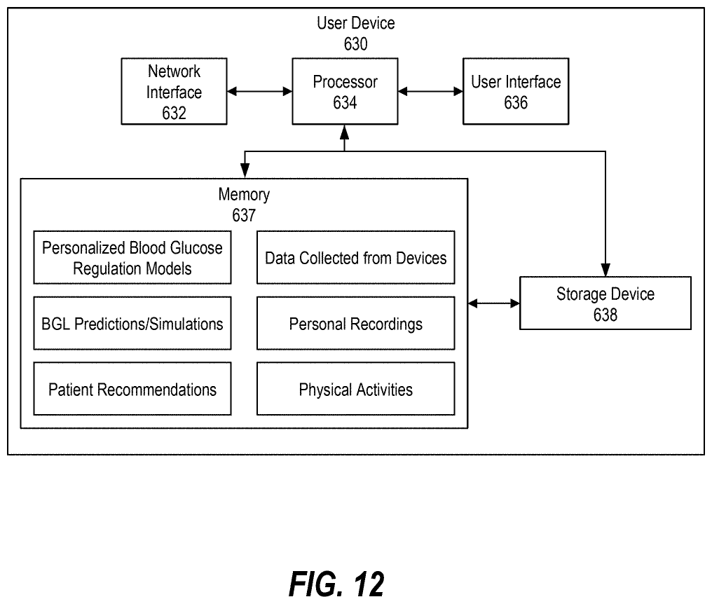

FIG. 12 is a schematic representation of an exemplary user device 630 for implementing method 10, according to some embodiments of the present disclosure. As shown in FIG. 12, in some embodiments, user device 630 includes a network interface 632, a processor 634, and a user interface 636. and a storage device. In some embodiments, user device 630 includes one or more storage devices, such as a memory 637 or a physical storage device 638 (e.g., hard drive, solid-state drive, etc.). Memory 637 may be a random-access memory (RAM) or a read-only memory (ROM). In some embodiments, memory 637 stores a set of instructions for instructing processor 634 to perform at least a part of the steps or processes of method 10. In some embodiments, memory 637 and/or storage device 638 store at least a part of the data collected from the individual, one or more models generated by method 10, BGL predictions or simulations, or personalized recommendations. User interface 636 may include a display and/or one or more input/output devices such as, for example, a touchscreen, a keyboard, a mouse, a track pad, and the like.

In some embodiments, data collection devices 640 may include one or more of, but not limited to, a glucometer 101 (e.g., continuous glucose monitors (CGM), blood glucose meters using test strips, and non-invasive blood glucose meters), an insulin delivery device (e.g., insulin delivery syringes, pens, pumps, or artificial pancreas systems), an activity tracking device 103 (e.g., fitness trackers, smart watches, or other wearable or implantable smart sensors), a user device 630 for receiving personal data recordings, or the like. The data collected from data collection devices 640 may include one or more of the nutritional intake data, blood glucose level measurements, insulin intake data, physical activity data, patient profile data, body index data, or demographics data described above.

In some embodiments, data collection terminals 650 may include one or more of, but not limited to, a pharmacy, a clinic, a hospital, a health care center, a medical office, a research institution, or the like where electronic health records (EHR) 106, personal health records (PHR), or other medical recordings (e.g., health or medical data) are collected.

### Exemplary Applications of Personalized Blood Glucose Regulation Model

As shown in FIG. 3, in some embodiments, method 10 includes blood glucose level prediction process 400. In some embodiments, blood glucose level prediction process 400 includes one or more steps to predict or simulate blood glucose levels over a period. In some embodiments, blood glucose level prediction process 400 includes one or more steps to predict or identify risks of blood glucose excursions. In some embodiments, blood glucose level prediction process 400 is performed by server 610 and the predicted or simulated BGL are sent to user device 630. In some embodiments, blood glucose level prediction process 400 is performed by user device 630.

In some embodiments, method 10 includes personalized recommendations generation process 500. In some embodiments, personalized recommendation generation process 500 may include one or more steps to provide personalized blood glucose regulation optimization strategies (e.g., bolus and/or basal insulin titration or therapy, nutritional plans, or physical activity plans), personalized preventive or corrective actions for an individual, and/or send signals, such as alerts or commands to various devices or systems (e.g., insulin delivery devices, driving systems, vehicles, machines, or equipment) to protect the safety of the individual and others. Processes 400 and 500 are described below.

Prediction and Simulation of Blood Glucose Levels and Identification of Risks of Glucose Excursions

In some embodiments, in process 400, the personalized blood glucose regulation model generated in process 300 is used to generate predictions of BGL of an individual at one or more time points over a period. In some embodiments, data of the individual are collected from user device 630, one or more data collection devices 640, and/or one or more data collection terminals 650 to generate at least one data set as input to the personalized blood glucose regulation model.

In some embodiments, in process 400, the personalized blood glucose regulation model generated in process 300 is used to generate a personalized simulation of a blood glucose curve for an individual over a period in the near future. For example, a simulated blood glucose curve over a period ranging from 1 hour to 6 hours may be generated. In some embodiments, the simulated blood glucose curve is used to identify risks of glucose excursions in the near future, such as identifying a risk of hypoglycemia or hyperglycemia in the next 2 hours. In some embodiments, based on the level of the identified risk of glucose exclusions and/or degree of the glucose exclusions, an alert or command is sent to the individual, a healthcare provider, an insulin delivery device, a driving system (e.g., an autonomous or assistive driving system), a vehicle, a machine, or an equipment, etc., as described below.

Personalized Recommendations for Blood Glucose Regulation

In some embodiments, the predictions of BGL or simulated blood glucose curve of an individual is used to titrate or optimize one or more personalized bolus and/or basal insulin doses for the individual to regulate the individual's blood glucose level within a target glucose range or to obtain a desired glucose curve for the individual. For example, to optimize bolus and/or basal insulin doses, a number of simulated blood glucose curves based on different combination of insulin molecules and doses (bolus and/or basal insulin) can be generated. The different combination of insulin molecules and doses can be compared and ranked based on one or more quantitative metrics of the simulated blood glucose curves. The one or more quantitative metrics may include one or more of the length of time interval during which the simulated BGL stays within a target glucose range (i.e., time in range (TIR)), the risks of hyperglycemic or hypoglycemic excursions within a time interval, the degree and/or duration of hyperglycemic or hypoglycemic excursions within a time interval, and the number and/or degree of fluctuations of BGL within a time interval. A combination of insulin molecules and doses that would result in a longer time interval of BGL within the target range, lower risk, degree, and/or duration of hyperglycemic or hypoglycemic excursions, and/or less BGL fluctuations may be ranked higher among the combinations.

In some embodiments, a combination of insulin molecules and doses (bolus and/or basal insulin) having the highest rank is recommended to the individual as an optimized insulin titration or insulin therapy. The recommended combination of insulin molecules and doses is determined based on the individual's data and the individual's metabolic and activity pattern represented by the personalized blood glucose regulation model, thereby providing a personalized recommendation for regulation blood glucose level to the individual. In some embodiments, the recommended combination of insulin molecules and doses can be provided to the individual shortly after receiving input data of the individual or upon receiving a request from the individual via the user interface, thereby allowing the individual to adjust insulin intake in a timely manner.

In some embodiments, the personalized blood glucose regulation model generated in process 300 is used to optimize a nutritional plan and/or a physical activity plan for an individual to obtain a desired glucose curve over a period. In some embodiments, the personalized blood glucose regulation model generated in process 300 is used to optimize a nutritional plan and/or a physical activity plan for an individual to lose weight or gain muscle increase. In some embodiments, a desired glucose curve may have a longer time interval of BGL within the target range (i.e., longer TIR), lower risk, level, and/or duration of hyperglycemic or hypoglycemic excursions, and/or less BGL fluctuations during the period. In some embodiments, the nutritional plan includes recommendation for the category, type, quantity, and/or time of food or nutrient intake over a period. In some embodiments, the physical activity plan includes recommendation for the type, duration, time, and/or level of physical activity over a period. In some embodiments, the recommended nutritional plan and/or a physical activity plan can be provided to the individual shortly after receiving input data of the individual or upon receiving a request from the individual via the user interface, thereby allowing the individual to adjust nutritional intake and/or physical activity in a timely manner.

Personalized Recommendations for Preventive or Corrective Actions

In some embodiments, the predictions of BGL or simulated blood glucose curve of an individual is used to generate one or more recommendations for preventive or corrective actions to the individual to reduce or prevent potential glucose excursions, increase TIR, and/or reduce fluctuations in BGL. Non-limiting examples of preventive or corrective actions include additional insulin doses or changes in insulin doses (bolus and/or basal), additional or changes in food or nutrient intake, and changes in physical activities. In some embodiments, recommendations for preventive or corrective actions are provided in response to change of the individual's behavior, such as change of exercise level, participating in sports, or eating a meal not typical to the individual's diet. In some embodiments, recommendations for preventive or corrective actions are provided upon the individual's request.

In some embodiments, the of BGL or simulated blood glucose curve of an individual is used to generate a signal, such as an alert (e.g., an alert of a potential severe glucose excursion) or a command. In some embodiments, the signal is provided to the individual, such as via user interface 636 of user device 630. The individual may also receive a recommendation for a taking preventive or corrective action along with the signal. In some embodiments, the signal is provided to an insulin delivery device. The insulin delivery device, upon receiving the signal, may adjust the insulin molecule, dose, and/or time of delivery of an insulin intake, or may deliver or skip an additional insulin dose. In some embodiments, the signal is provided to an apparatus that the individual is operating, such as a vehicle, an airplane, a machine, or an equipment, or an operating system thereof (e.g., an autonomous or assistive driving system or application, a driver safety system, or a driving command center or network). In some embodiments, the operating system may stop the apparatus according to a safe procedure or taking control of the apparatus. For example, in response to receiving an alert of a potential severe hypoglycemic excursion (e.g., a high risk, long duration, or high degree), an autonomous driving system may take control of the vehicle the individual is operating to protect the safety of the individual and prevent potential accidents.

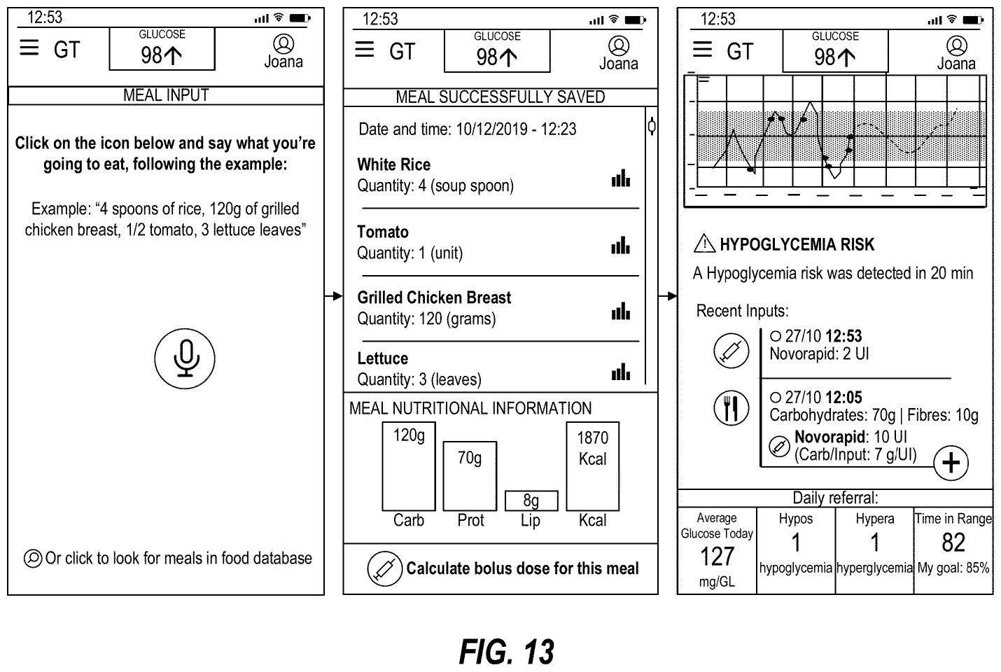

FIG. 13 is a graphical representation of an exemplary user interface 636 on an exemplary user device 630 for predicting blood glucose levels, according to some embodiments of the present disclosure. FIG. 13 depicts a sequential transition of three images of user interface 636, illustrating the receiving and recording of an individual's input of nutritional intake data, providing a simulated glucose curve, and identifying a potential hyperglycemic risk in the near future via user interface 636.

### Exemplary Predictions or Simulations of Blood Glucose Levels

Personalized glucose regulation models generated in accordance with the present disclosure are used to generate simulated blood glucose levels (BGL) and blood insulin levels (BIL) using different exemplary input data sets over different periods.

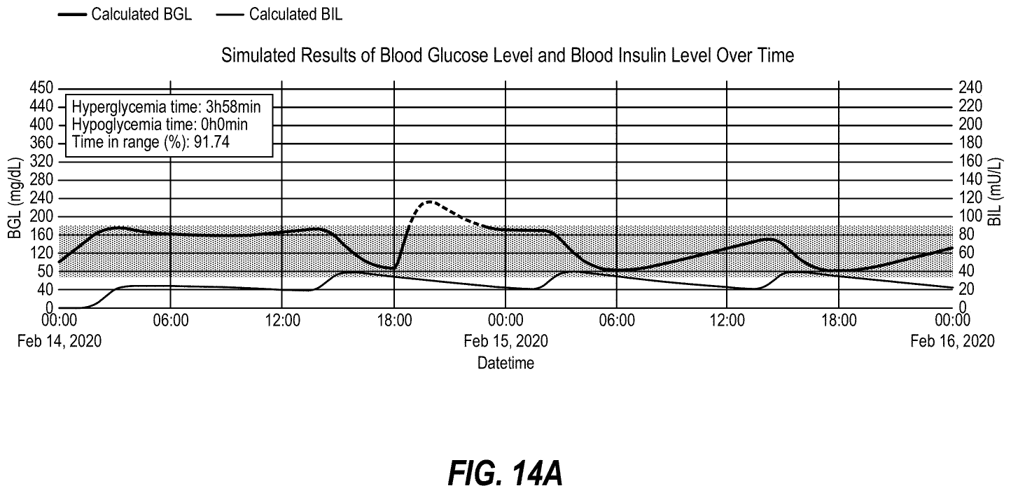

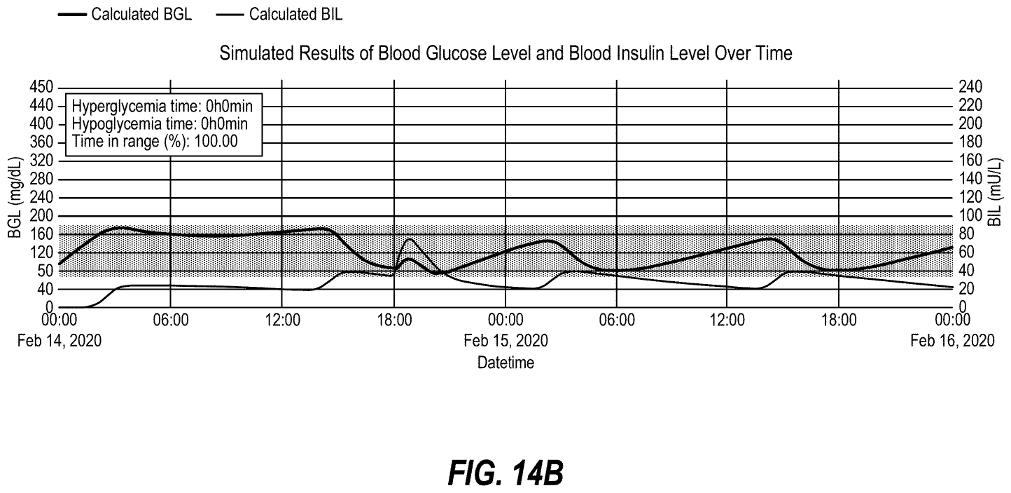

FIG. 14A is a graphical representation of simulated blood glucose levels and blood insulin levels of an exemplary diabetic individual after eating a meal with only carbohydrates without bolus insulin injection, according to some embodiments of the present disclosure. The meal is taken at 18:00 on Feb. 14, 2020, and contains 25.7 g carbohydrate. A dose of basal insulin is taken by the individual every six hours starting from 00:00 on Feb. 14, 2020. As shown in FIG. 14A, the simulated results suggest a potential hyperglycemia excursion over a period of about 4 hours after the meal.

FIG. 14B is a graphical representation of simulated blood glucose levels and blood insulin levels of the exemplary diabetic individual of FIG. 14A after eating the same meal with only carbohydrates and injecting a dose of bolus insulin, according to some embodiments of the present disclosure. The dose of bolus insulin is recommended to the individual to prevent the hyperglycemia in accordance with embodiments of the present disclosure. As shown in FIG. 14B, the simulated results suggest no hyperglycemia excursion after the meal.

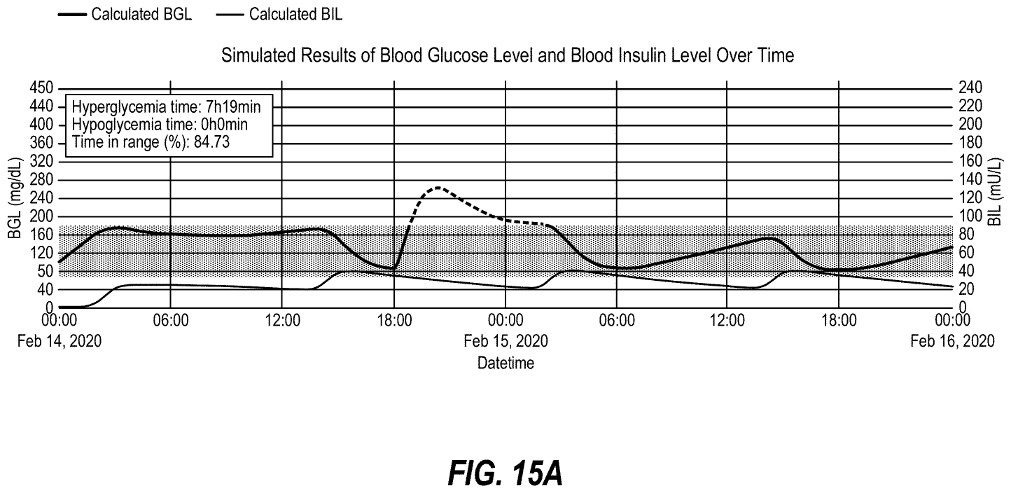

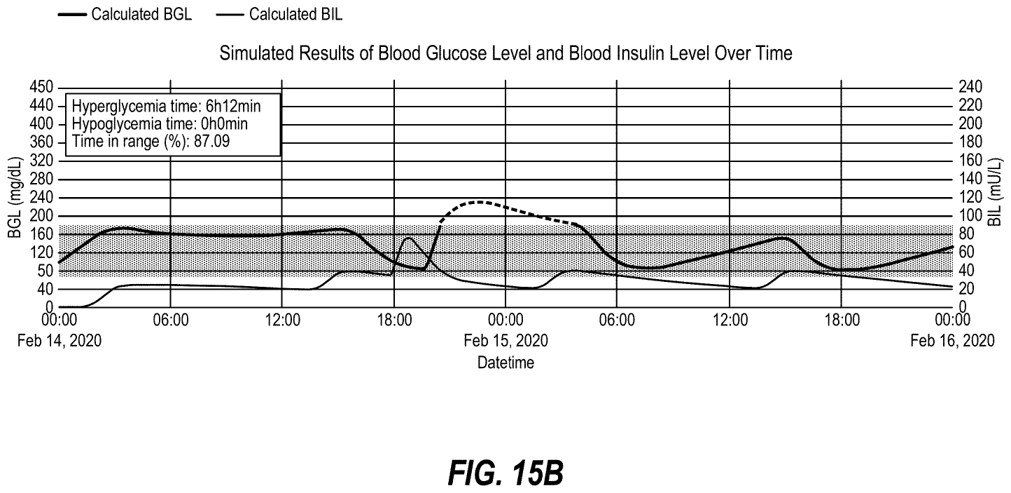

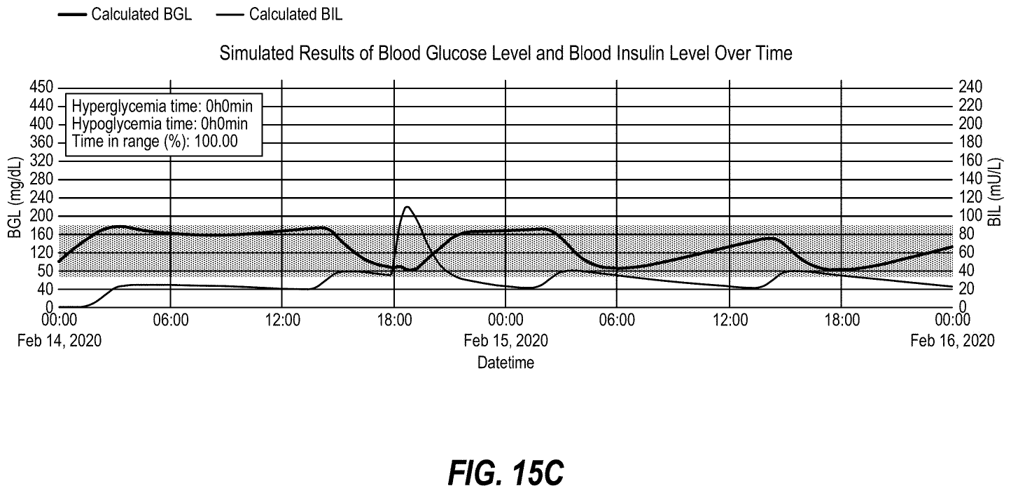

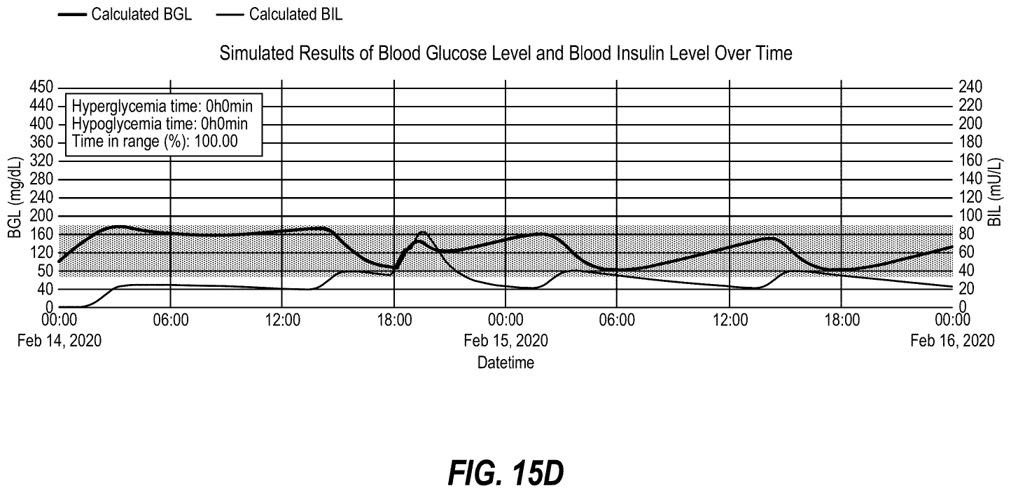

FIG. 15A is a graphical representation of simulated blood glucose levels and blood insulin levels of an exemplary diabetic individual after eating a meal containing carbohydrate, protein, and lipid without bolus insulin injection, according to some embodiments of the present Disclosure. The meal is taken at 18:00 on Feb. 14, 2020, and contains 25.7 g carbohydrate, 13.4 g protein, and 4.7 g lipid. A dose of basal insulin is taken by the individual every six hours starting from 00:00 on Feb. 14, 2020. As shown in FIG. 15A, the simulated results suggest a potential hyperglycemia excursion over a period of about 7 hours after the meal.

FIG. 15B is a graphical representation of simulated blood glucose levels and blood insulin levels of an exemplary diabetic individual after eating the meal containing carbohydrate, protein, and lipid with injection of a dose of bolus insulin shortly before the meal, according to some embodiments of the present disclosure. The dose of the bolus insulin is calculated based only on the intake of carbohydrate using the carbohydrate counting method and injected at 17:45 on Feb. 14, 2020. As shown in FIG. 15B, the bolus dose corrects the short-term hyperglycemic effect caused by carbohydrate intake, but not the long-term hyperglycemia effect caused by protein and lipid intake.

FIG. 15C is a graphical representation of simulated blood glucose levels and blood insulin levels of an exemplary diabetic individual after eating the meal containing carbohydrate, protein, and lipid with injection of a higher dose of bolus insulin shortly before the meal, according to some embodiments of the present disclosure. The higher dose of bolus insulin is recommended to the individual to prevent the hyperglycemia caused by carbohydrate, protein, and lipid intake in accordance with embodiments of the present disclosure and is injected at 17:45 on Feb. 14, 2020. As shown in FIG. 15C, the bolus insulin dose corrects both the short and long term hyperglycemic effect caused by carbohydrate, protein, and lipid intake, thereby improving the TIR and glucose control.

FIG. 15D is a graphical representation of simulated blood glucose levels and blood insulin levels of the exemplary diabetic individual of FIG. 15A after eating the meal containing carbohydrate, protein, and lipid with the injection of two doses of bolus insulin at different times, according to some embodiments of the present disclosure. The first dose of bolus insulin is injected at 17:45 on Feb. 14, 2020, and the second dose of bolus insulin is injected at 18:45 on Feb. 14, 2020. As shown in FIG. 15D, the two doses of bolus insulin correct both the short and long-term hyperglycemic effects caused by carbohydrate, protein, and lipid intake, improving the TIR and glucose control with lower hypoglycemia risk than taking one dose of bolus insulin.

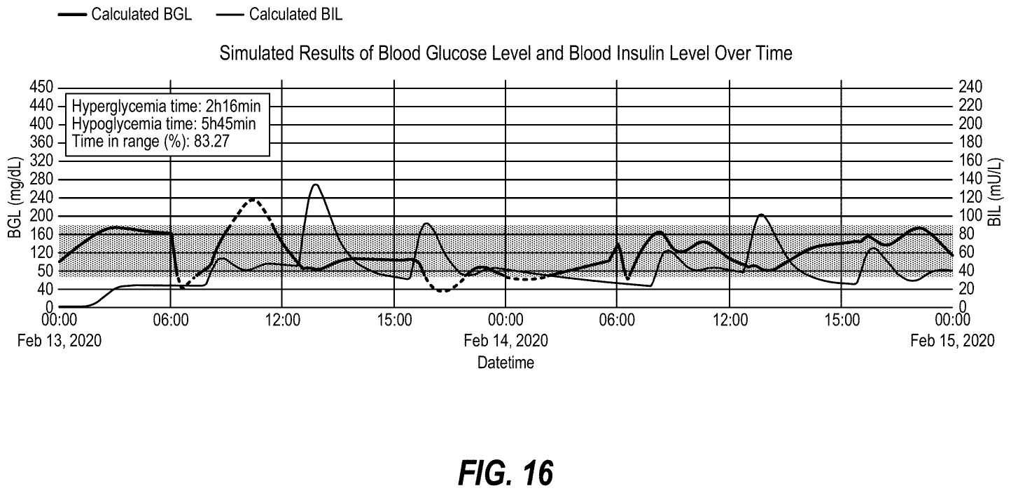

FIG. 16 is a graphical representation of simulated blood glucose levels and blood insulin levels of an exemplary diabetic individual generated by an exemplary personalized blood glucose regulation model, according to some embodiments of the present disclosure. The input data used for generating the simulated results are shown in the tables below.

TABLE 1
Exemplary meals of an exemplary diabetic individual.

| Date | Time | Carbohydrate (g) | Protein (g) | Lipid (g) |
|---|---|---|---|---|
| 2020 Feb. 14 | 8:00 | 10 | 26.88 | 5 |
| 2020 Feb. 14 | 13:00 | 25 | 40.32 | 15 |
| 2020 Feb. 14 | 19:00 | 20 | 20.16 | 2.1 |
| 2020 Feb. 13 | 8:00 | 25.5 | 42 | 4.1 |
| 2020 Feb. 13 | 13:00 | 35.74 | 53.76 | 5.6 |
| 2020 Feb. 13 | 19:00 | 9.48 | 20.16 | 8.4 |
| 2020 Feb. 14 | 5:30 | 15 | 10 | 2.1 |

TABLE 2
Exemplary bolus insulin intake of
an exemplary diabetic individual.

| Date | Time | Insulin | Dose (U) |
|---|---|---|---|
| 2020 Feb. 14 | 7:45 | Humalog ® | 2 |
| 2020 Feb. 14 | 12:45 | Humalog ® | 3 |
| 2020 Feb. 14 | 18:45 | Humalog ® | 2 |
| 2020 Feb. 13 | 7:45 | Humalog ® | 1.5 |
| 2020 Feb. 13 | 12:45 | Humalog ® | 4 |
| 2020 Feb. 13 | 18:45 | Humalog ® | 3 |

TABLE 3
Exemplary basal insulin intake of an exemplary diabetic individual.

| Date | Time | Insulin | Dose (U) |
|---|---|---|---|
| 2020 Feb. 13 | 8:00 | Lantus ® | 10 |
| 2020 Feb. 13 | 20:00 | Lantus ® | 10 |
| 2020 Feb. 14 | 8:00 | Lantus ® | 10 |
| 2020 Feb. 14 | 20:00 | Lantus ® | 10 |
| 2020 Feb. 13 | 0:00 | Lantus ® | 10 |

TABLE 4
Exemplary physical activity of an exemplary diabetic individual.

| Date | Time | Duration (minutes) | Average BPM |
|---|---|---|---|
| 2020 Feb. 13 | 6:00 | 30 | 150 |
| 2020 Feb. 14 | 6:00 | 30 | 150 |

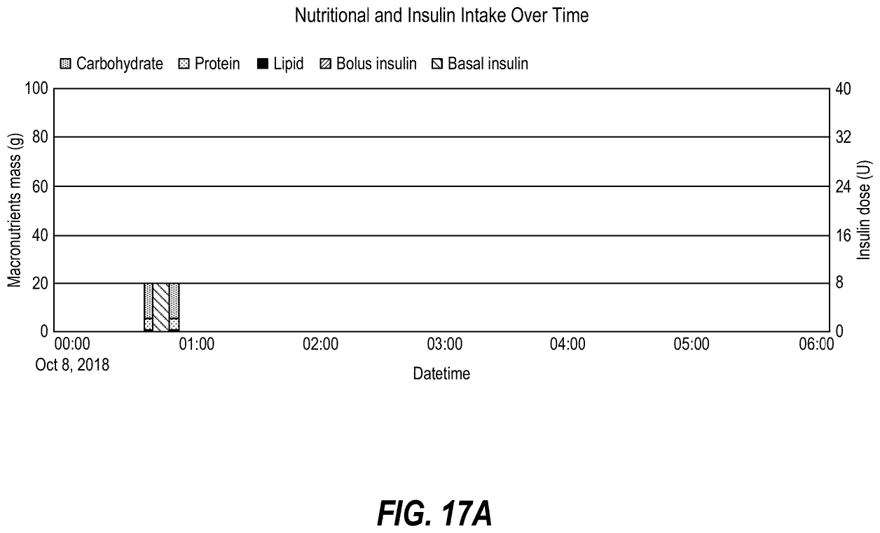

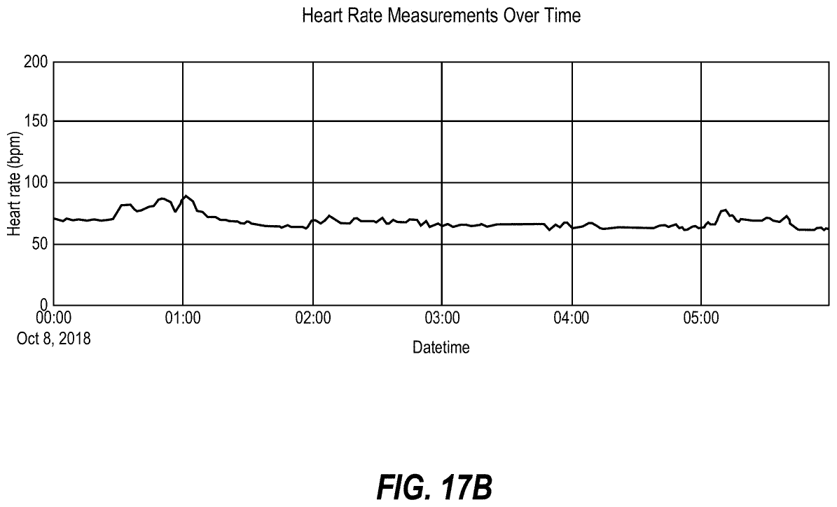

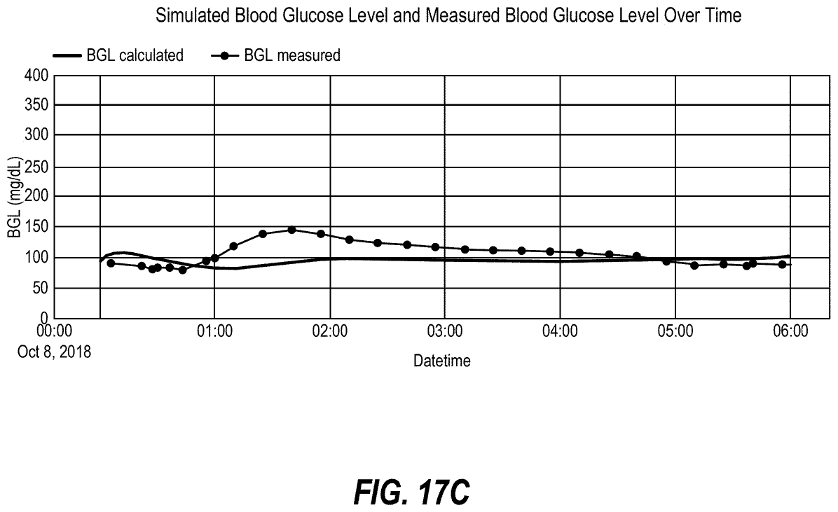

FIG. 17A is a graphical representation of exemplary nutritional intake of an exemplary diabetic individual in dawn period, according to some embodiments of the present disclosure. FIG. 17B is a graphical representation of exemplary heart rate measurements of the exemplary diabetic individual of FIG. 17A in the same dawn period, according to some embodiments of the present disclosure. FIG. 17C is a graphical representation of measured BGL and simulated BGL of the exemplary diabetic individual of FIG. 17A in the same drawn period. The simulated BGL was generated using an exemplary blood glucose regulation model according to some embodiments of the present disclosure.

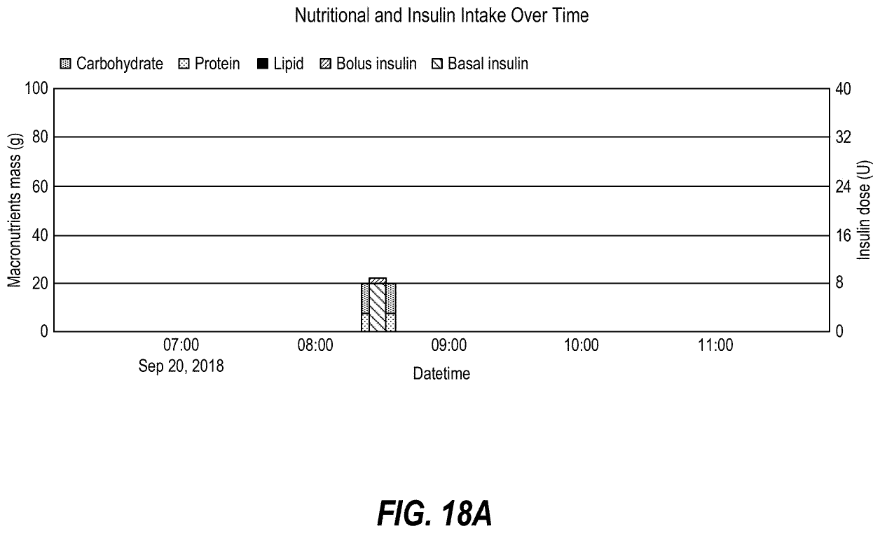

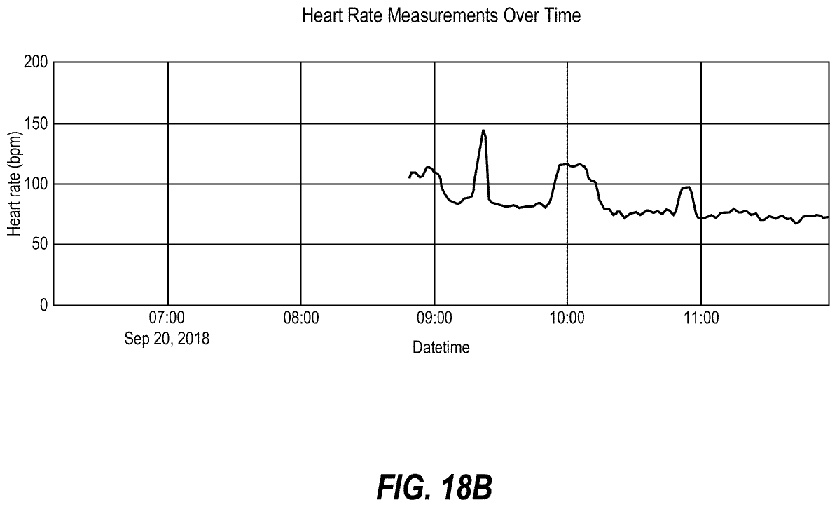

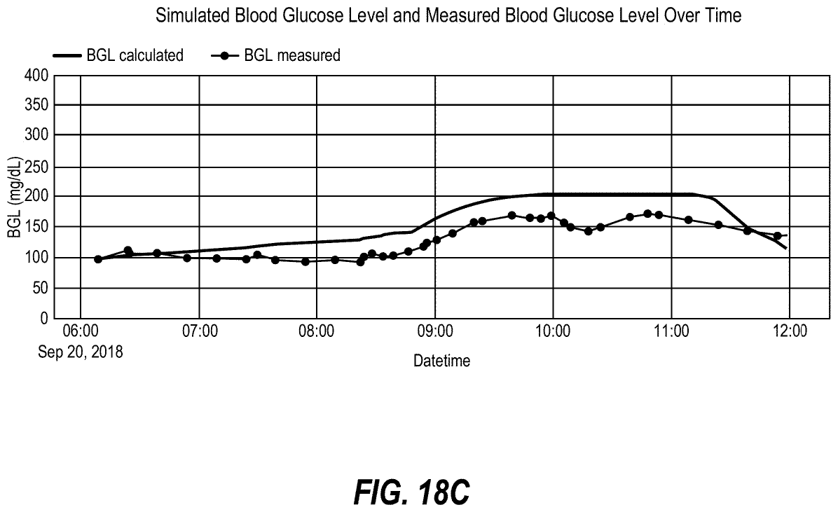

FIG. 18A is a graphical representation of exemplary nutritional intake of an exemplary diabetic individual in a morning period, according to some embodiments of the present disclosure. FIG. 18B is a graphical representation of exemplary heart rate measurements of the exemplary diabetic individual of FIG. 18A in the same morning period, according to some embodiments of the present disclosure. FIG. 18C is a graphical representation of measured BGL and simulated BGL of the exemplary diabetic individual of FIG. 18A in the same morning period. The simulated BGL was generated using an exemplary blood glucose regulation model according to some embodiments of the present disclosure.

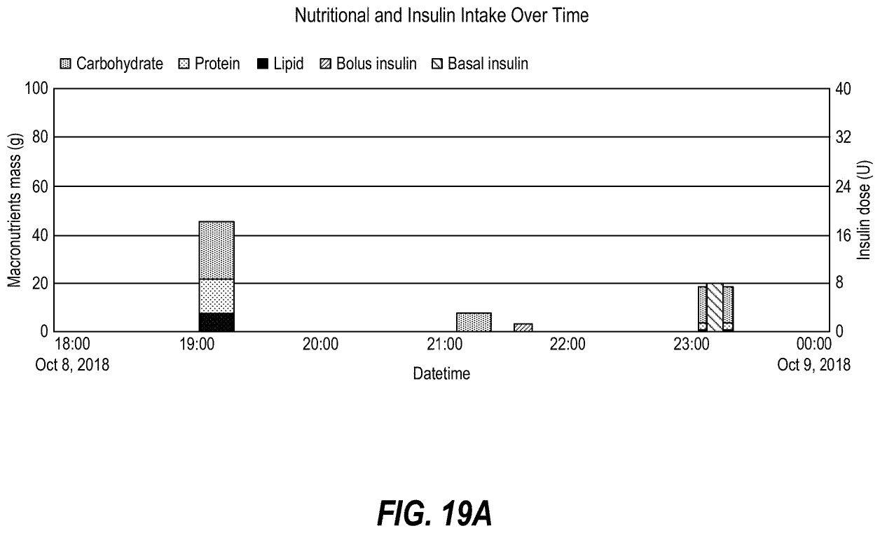

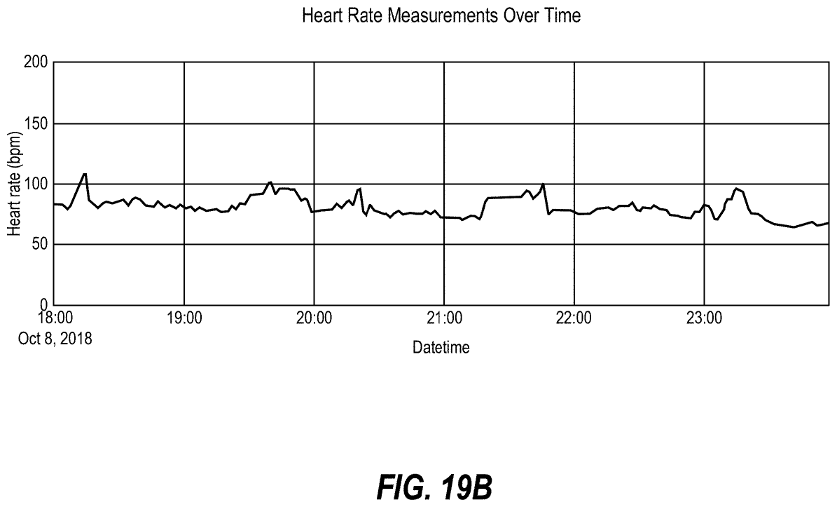

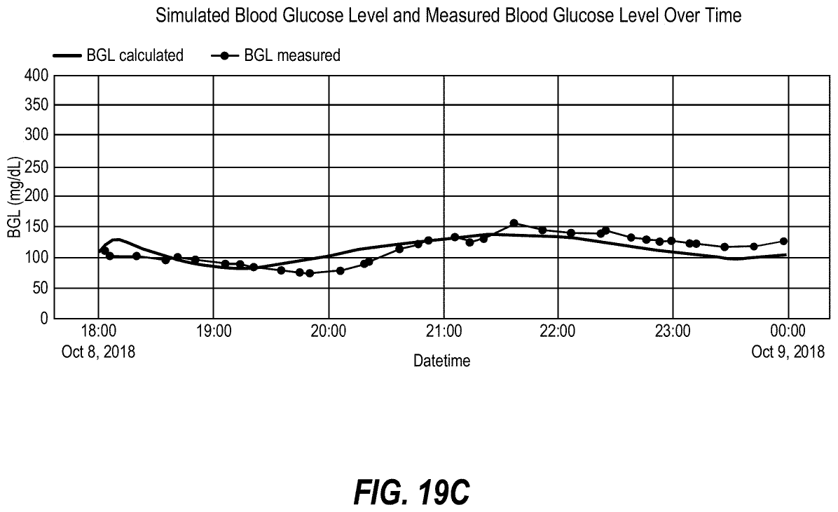

FIG. 19A is a graphical representation of exemplary nutritional intake of an exemplary diabetic individual in a night period, according to some embodiments of the present disclosure. FIG. 19B is a graphical representation of exemplary heart rate measurements of the exemplary diabetic individual of FIG. 19A in the same night period, according to some embodiments of the present disclosure. FIG. 19C is a graphical representation of measured BGL and simulated BGL of the exemplary diabetic individual of FIG. 19A in the same night period. The simulated BGL was generated using an exemplary blood glucose regulation model according to some embodiments of the present disclosure.

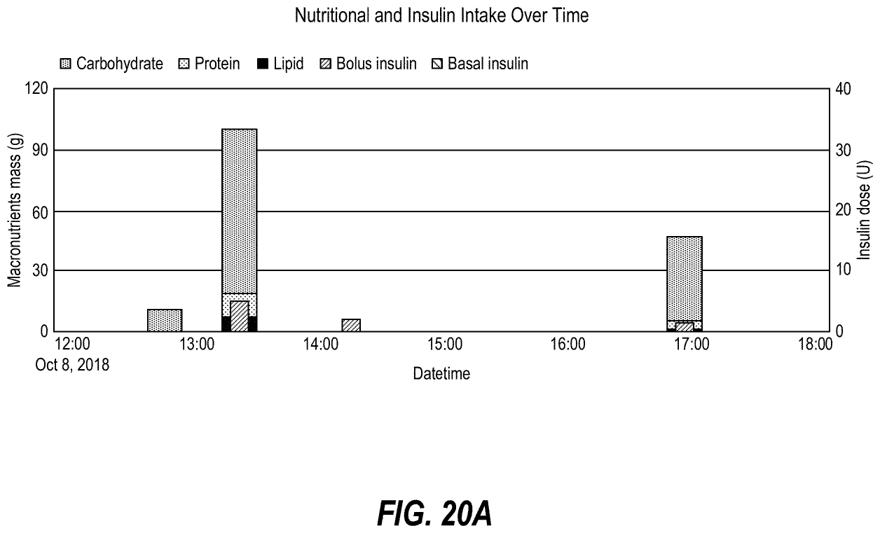

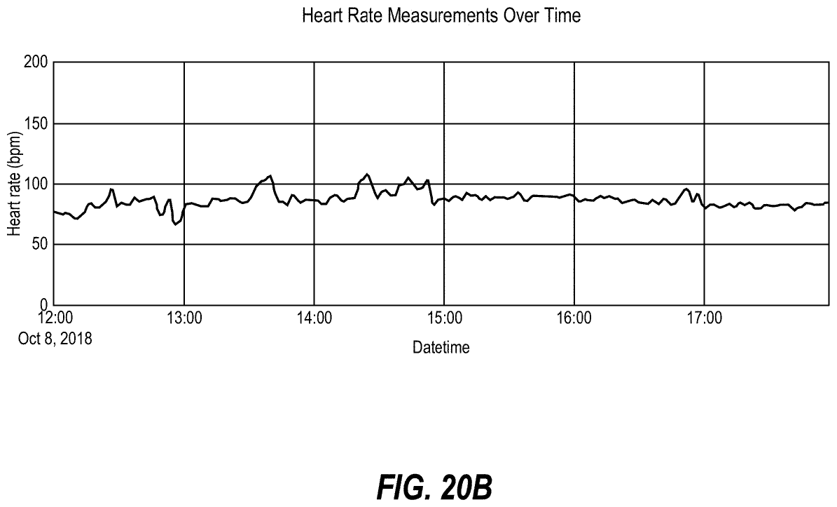

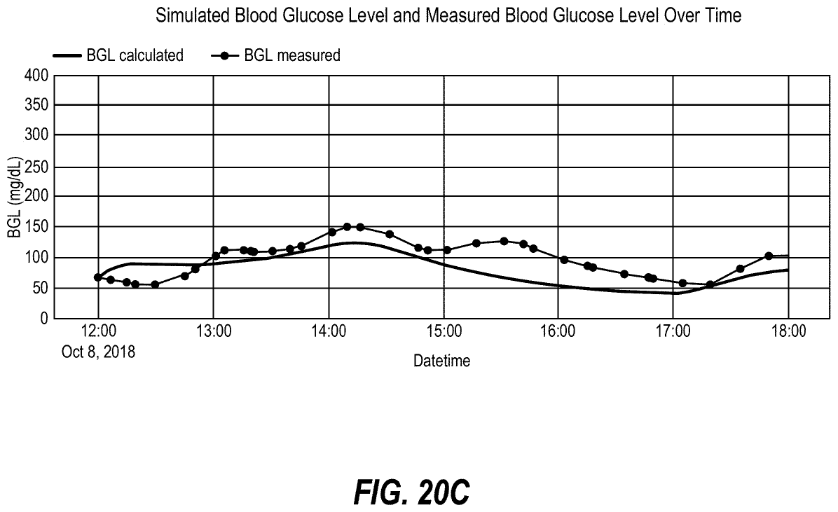

FIG. 20A is a graphical representation of exemplary nutritional intake of an exemplary diabetic individual in an afternoon period, according to some embodiments of the present disclosure. FIG. 20B is a graphical representation of exemplary heart rate measurements of the exemplary diabetic individual of FIG. 20A in the same afternoon period, according to some embodiments of the present disclosure. FIG. 20C is a graphical representation of measured blood glucose levels and simulated blood glucose levels of the exemplary diabetic individual of FIG. 20A in the same afternoon period. The simulated BGL was generated using an exemplary blood glucose regulation model according to some embodiments of the present disclosure.

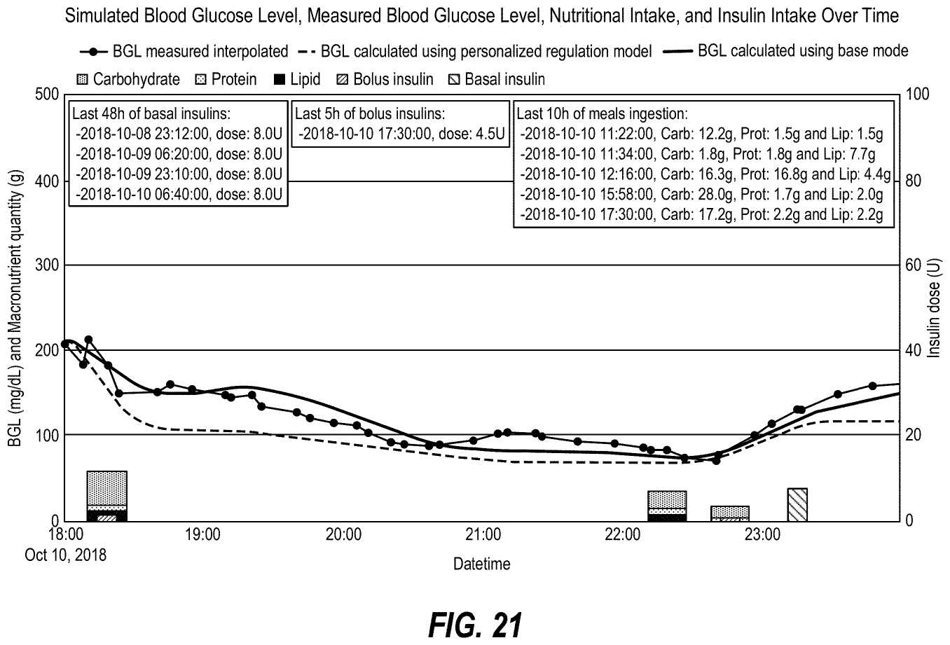

FIG. 21 is a graphical representation of exemplary nutritional intake, insulin intake, measured blood glucose levels, and two exemplary simulated glucose curves of an exemplary diabetic individual, according to some embodiments of the present disclosure. As shown in FIG. 20, a first exemplary simulated glucose curve is generated by a personalized glucose regulation model for the individual determined in process 320A, according to embodiments of the present disclosure. A second exemplary simulated glucose curve is generated by a populational blood glucose regulation base model of the individual determined in process 350A, according to embodiments of the present disclosure. As shown in FIG. 20, the simulated glucose curves trace the measured blood glucose levels with different accuracy. As described above, in some embodiments, a regulation model with better accuracy is selected for predicting blood glucose levels for the individual.

While the present disclosure has been shown and described referring to particular embodiments thereof, it will be understood that the present disclosure can be practiced, without modification, in other environments. The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments.

Embodiments of the present disclosure may be embodied as a method, a process, a device, a system, a computer program product, etc. Accordingly, embodiments of the present disclosure may take the form of an entirely hardware embodiment, an entirely software embodiment, or an embodiment combining software and hardware for allowing a specialized device having the described specialized components to perform the functions described above.

Furthermore, embodiments of the present disclosure may take the form of a computer program product embodied in one or more computer-readable storage media that may be used for storing computer-readable program codes. Based on such an understanding, the technical solutions of the present disclosure can be implemented in a form of a software product. The software product can be stored in a non-volatile storage medium (which can be a CD-ROM, a USB flash memory, a mobile hard disk, and the like). The storage medium can include a set of instructions for instructing a computer device (which may be a personal computer, a server, a network device, a mobile device, or the like) or a processor to perform a part of the steps of the methods provided in the embodiments of the present disclosure.

The foregoing storage medium may include, for example, any medium that can store a program code, such as a USB flash disk, a removable hard disk, a Read-Only Memory (ROM), or a Random-Access Memory (RAM). The storage medium can be a non-transitory computer-readable medium. Exemplary forms of non-transitory media include a hard disk, a solid state drive, a ROM, a CD-ROM, any other optical data storage medium, any physical medium with patterns of holes, a RAM, a PROM, an EPROM, a FLASH-EPROM or any other flash memory, NVRAM any other memory chip or cartridge, and networked versions of the same.

Computer programs based on the written description and disclosed methods are within the skill of an experienced developer. Various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, C++, Objective-C, HTML, HTML/AJAX combinations, XML, or HTML with included Java applets.

It should be noted that, as used in this specification and the appended claims, the singular forms “a,” “an,” and “the,” and any singular use of any word, include plural referents unless expressly and unequivocally limited to one referent. As used herein, the terms “include,” “comprise,” “contain,” and their grammatical variants are intended to be non-limiting, such that recitation of items in a list is not to the exclusion of other like items that can be substituted or added to the listed items. The term “if” may be construed as “at the time of” “when,” “in response to,” or “in response to determining.”

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps or processes of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

This description and the accompanying drawings that illustrate exemplary embodiments should not be taken as limiting. Various structural, electrical, and operational changes may be made without departing from the scope of this description and the claims, including equivalents. In some instances, well-known structures and techniques have not been shown or described in detail so as not to obscure the disclosure. Similar reference numbers in two or more figures represent the same or similar elements. Furthermore, elements and their associated features that are disclosed in detail referring to one embodiment may, whenever practical, be included in other embodiments in which they are not specifically shown or described. For example, if an element is described in detail referring to one embodiment and is not described referring to a second embodiment, the element may nevertheless be claimed as included in the second embodiment.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A computer-implemented method for optimizing blood glucose level regulation, the method comprising:
collecting a first plurality of data sets associated with an individual from a database, the first plurality of data sets comprising
nutritional intake data at a plurality of time points over a first period, the nutritional intake data comprising carbohydrate intake, protein intake, and lipid intake;
blood glucose level measurements at a plurality of time points over the first period, and
insulin intake data at a plurality of time points over the first period;
generating a personalized blood glucose regulation model for the individual, comprising
creating a first training data subset, a first validation data subset, and a first test data subset from the first plurality of data sets;
determining a plurality of parameters of the personalized blood glucose regulation model using an optimization algorithm, the first training data subset, and the first validation data subset; and
determining a first evaluation metric of the personalized blood glucose regulation model using the first test data subset;
determining an approved personalized blood glucose regulation model based on the first evaluation metric; and
determining a working personalized blood glucose regulation model based on the approved personalized blood glucose regulation model;
receiving a second plurality of data sets associated with the individual, the second plurality of data sets comprising
nutritional intake data at a plurality of time points over a second period, the nutritional intake data comprising at least one of carbohydrate intake, protein intake, or lipid intake; and
insulin intake data over the second period;
generating predicted blood glucose levels at one or more time points after the second period using the working personalized blood glucose regulation model and the second plurality of data sets; and
providing an instruction for regulating the individual's blood glucose level based on the generated predicted blood glucose levels.

2. The method of claim 1, wherein the insulin intake data of the first plurality of data sets or the second plurality of data sets comprise at least one of an amount of a bolus insulin intake, an amount of a basal insulin intake, a type of insulin molecules of a bolus insulin intake, a type of insulin molecules of a basal insulin intake.

3. The method of claim 1, wherein the nutritional intake data over the first period or the second period further comprise at least one of fibre intake, category of food, type of food, or calorie intake.

4. The method of claim 1, wherein the first plurality of data sets or the second plurality of data sets further comprise at least one of
physical activity data at a plurality of time points during the first period or the second period, the physical activity data comprising at least one of heart rate measurements, calories or kilocalories burned, steps, type of activity, duration of activity, Metabolic Equivalent of Task (MET), duration of sleep, or phase of sleep;
profile data comprising at least one of sex, birth date, type of diabetes, or drug therapy; or
body index data at least one time point over the first period, the body index data comprising at least one of weight, height, or body mass index (BMI).

5. The method of claim 1, further comprising
collecting a third plurality of data sets associated with a group of individuals sharing one or more characteristics from the database, the third plurality of data sets comprising
nutritional intake data at a plurality of time points over the first period, the nutritional intake data comprising at least one of carbohydrate intake, protein intake, or lipid intake; and
insulin intake data at a plurality of time points over the first period;
generating a populational blood glucose regulation base model comprising
creating a second training data subset, a second validation data subset, and a second test data subset based on the third plurality of data sets;
determining a plurality of parameters of the populational blood glucose regulation base model using an optimization algorithm, the second training data subset, and the second validation data subset;
determining a second evaluation metric of the populational blood glucose regulation base model using the second test data subset; and
determining an approved populational blood glucose regulation base model based on the second evaluation metric.

6. The method of claim 5, further comprising
determining a third evaluation metric for the approved populational blood glucose regulation base model using the first test data subset; and
determining the personalized blood glucose regulation model or the populational blood glucose regulation base model as the working personalized blood glucose regulation model based on the second and third evaluation metrics.

7. The method of claim 1, further comprising
collecting a third plurality of data sets associated with the individual from the database, the third plurality of data sets comprising
nutritional intake data at a plurality of time points over a third period, the nutritional intake data comprising carbohydrate intake, protein intake, and lipid intake,
blood glucose level measurements at a plurality of time points over the third period, and
insulin intake data at a plurality of time points over the third period;
generating predicted blood glucose levels at a plurality of time points over a first time interval of the third period using the personalized blood glucose regulation model and the third plurality of data sets;
creating a second training data set, a second validation data set, and a second test data set using the third plurality of data sets and the predicted blood glucose levels at the plurality of time points over the first time interval;
training a machine learning model using the second training data set and the second validation data set;
determining a second evaluation metric of the machine learning model using the second test data set;
generating a personalized hybrid model comprising the personalized blood glucose regulation model and the machine learning model; and
determining the personalized hybrid model as the working personalized blood glucose regulation model.

8. A non-transitory computer-readable storage medium storing a set of instructions that, when executed by one or more processors, cause the one or more processors to perform a method of predicting blood glucose levels, the method comprising:
collecting a first plurality of data sets associated with an individual from a database, the first plurality of data sets comprising
nutritional intake data at a plurality of time points over a first period, the nutritional intake data comprising carbohydrate intake, protein intake, and lipid intake;
blood glucose level measurements at a plurality of time points over the first period, and
insulin intake data at a plurality of time points over the first period;
generating a personalized blood glucose regulation model for the individual, comprising
creating a first training data subset, a first validation data subset, and a first test data subset based on the first plurality of data sets;
determining a plurality of parameters of the personalized blood glucose regulation model using an optimization algorithm, the first training data subset, and the first validation data subset; and
determining a first evaluation metric of the personalized blood glucose regulation model using the first test data subset;
determining an approved personalized blood glucose regulation model based on the first evaluation metric; and
determining a working personalized blood glucose regulation model based on the approved personalized blood glucose regulation model;
receiving a second plurality of data sets associated with the individual, the second plurality of data sets comprising
nutritional intake data at a plurality of time points over a second period, the nutritional intake data comprising at least one of carbohydrate intake, protein intake, or lipid intake; and
insulin intake data over the second period;
generating predicted blood glucose levels at one or more time points after the second period using the working personalized blood glucose regulation model and the second plurality of data sets; and
providing an instruction for regulating the individual's blood glucose level based on the predicted blood glucose levels.

9. The medium of claim 8, wherein the insulin intake data of the first plurality of data sets or the second plurality of data sets comprise at least one of an amount of a bolus insulin intake, an amount of a basal insulin intake, a type of insulin molecules of a bolus insulin intake, a type of insulin molecules of a basal insulin intake.

10. The medium of claim 8, wherein the nutritional intake data over the first period or the second period further comprise at least one of fibre intake, category of food, type of food, or calorie intake.

11. The medium of claim 8, wherein the first plurality of data sets or the second plurality of data sets further comprise at least one of
physical activity data at a plurality of time points during the first period or the second period, the physical activity data comprising at least one of heart rate measurements, calories or kilocalories burned, steps, type of activity, duration of activity, Metabolic Equivalent of Task (MET), duration of sleep, or phase of sleep;
profile data comprising at least one of sex, birth date, type of diabetes, or drug therapy; or
body index data at least one time point over the first period, the body index data comprising at least one of weight, height, or body mass index (BMI).

12. The medium of claim 8, wherein the set of instructions, when executed by the one or more processors, cause the one or more processors to further perform:
collecting a third plurality of data sets associated with the individual from the database, the third plurality of data sets comprising
nutritional intake data at a plurality of time points over a third period, the nutritional intake data comprising carbohydrate intake, protein intake, and lipid intake,
blood glucose level measurements at a plurality of time points over the third period, and
insulin intake data at a plurality of time points over the third period;
generating predicted blood glucose levels at a plurality of time points over a first time interval of the third period using the personalized blood glucose regulation model and the third plurality of data sets;
creating a second training data set, a second validation data set, and a second test data set using the third plurality of data sets and the predicted blood glucose levels at the plurality of time points over the first time interval;
training a machine learning model using the second training data set and the second validation data set;
determining a second evaluation metric of the machine learning model using the second test data set;
generating a personalized hybrid model comprising the personalized blood glucose regulation model and the machine learning model; and
determining the personalized hybrid model as the working personalized blood glucose regulation model.

13. The medium of claim 12, wherein the set of instructions, when executed by the one or more processors, cause the one or more processors to further perform
determining a third evaluation metric for the approved populational blood glucose regulation base model using the first test data subset; and
determining the personalized blood glucose regulation model or the populational blood glucose regulation base model as the working personalized blood glucose regulation model based on the second and third evaluation metrics.

14. The medium of claim 8, wherein the set of instructions, when executed by the one or more processors, cause the one or more processors to further perform
collecting a third plurality of data sets associated with the individual from the database, the third plurality of data sets comprising
nutritional intake data at a plurality of time points over a third period before the first period, the nutritional intake data comprising carbohydrate intake, protein intake, and lipid intake, and
insulin intake data at a plurality of time points over the third period;
generating predicted blood glucose levels at a plurality of time points over the first period using the personalized blood glucose regulation model and the third plurality of data sets;
creating a second training data subset, a second validation data subset, and a second test data subset using the first plurality of data sets and the predicted blood glucose levels at the plurality of time points over the first period;
training a machine learning model using the second training data subset and the second validation data subset;
determining a second evaluation metric of the machine learning model using the second test data subset;
generating a personalized hybrid model comprising the personalized blood glucose regulation model and the machine learning model; and
determining the personalized hybrid model as the working personalized blood glucose regulation model.

15. A computer-implemented method of optimizing blood glucose level regulation, comprising:
receiving a personalized blood glucose regulation model for an individual from a remote server over a network, the remote server comprising
a non-transitory computer-readable storage medium storing a set of instructions that, when executed by the remote server, cause the remote server to perform a method for generating the personalized blood glucose regulation model, the method for generating the personalized blood glucose regulation model comprising
collecting a first plurality of data sets associated with the individual from a database, the first plurality of data sets comprising
nutritional intake data at a plurality of time points over a first period, the nutritional intake data comprising carbohydrate intake, protein intake, and lipid intake;
blood glucose level measurements at a plurality of time points over the first period, and
insulin intake data at a plurality of time points over the first period; and
creating a first training data subset, a first validation data subset, and a first test data subset based on the first plurality of data sets;
determining a plurality of parameters of the personalized blood glucose regulation model using an optimization algorithm, the first training data subset, and the first validation data subset; and
determining a first evaluation metric of the personalized blood glucose regulation model using the first test data subset; and
determining an approved personalized blood glucose regulation model based on the first evaluation metric;
receiving a second plurality of data sets associated with the individual, the second plurality of data sets comprising
nutritional intake data at a plurality of time points over a second period, the nutritional intake data comprising at least one of carbohydrate intake, protein intake, or lipid intake; and
insulin intake data over the second period;
generating predicted blood glucose levels at one or more time points after the second period using the personalized blood glucose regulation model and the second plurality of data sets; and.
providing an instruction for regulating the individual's blood glucose level based on the predicted blood glucose levels.

16. The method of claim 15, wherein the insulin intake data of the first plurality of data sets or the second plurality of data sets comprise at least one of an amount of a bolus insulin intake, an amount of a basal insulin intake, a type of insulin molecules of a bolus insulin intake, a type of insulin molecules of a basal insulin intake.

17. The method of claim 15, wherein the nutritional intake data over the first period or the second period further comprise at least one of fibre intake, category of food, type of food, or calorie intake.

18. The method of claim 15, wherein the first plurality of data sets or the second plurality of data sets further comprise at least one of
physical activity data at a plurality of time points during the first period or the second period, the physical activity data comprising at least one of heart rate measurements, calories or kilocalories burned, steps, type of activity, duration of activity, Metabolic Equivalent of Task (MET), duration of sleep, or phase of sleep;
profile data comprising at least one of sex, birth date, type of diabetes, or drug therapy; or
body index data at least one time point over the first period, the body index data comprising at least one of weight, height, or body mass index (BMI).

19. The method of claim 15, wherein the set of instructions, when executed by the remote server, cause the remote server to further perform
collecting a third plurality of data sets associated with a group of individuals a group of individuals sharing one or more characteristics from the database, the third plurality of data sets comprising
nutritional intake data at a plurality of time points over the first period, the nutritional intake data comprising at least one of carbohydrate intake, protein intake, or lipid intake; and
insulin intake data at a plurality of time points over the first period;
generating a populational blood glucose regulation base model comprising
creating a second training data subset, a second validation data subset, and a second test data subset based on the third plurality of data sets;
determining a plurality of parameters of the populational blood glucose regulation base model using an optimization algorithm, the second training data subset, and the second validation data subset;
determining a second evaluation metric of the populational blood glucose regulation base model using the second test data subset; and
determining an approved populational blood glucose regulation base model based on the second evaluation metric.

20. The method of claim 15, wherein the set of instructions, when executed by the remote server, cause the remote server to further perform
determining a third evaluation metric for the approved populational blood glucose regulation base model using the first test data subset; and
determining the personalized blood glucose regulation model or the populational blood glucose regulation base model as the working personalized blood glucose regulation model based on the second and third evaluation metrics.

21. The method of claim 15, wherein the set of instructions, when executed by the remote server, cause the remote server to further perform
collecting a third plurality of data sets associated with the individual from the database, the third plurality of data sets comprising
nutritional intake data at a plurality of time points over a third period, the nutritional intake data comprising carbohydrate intake, protein intake, and lipid intake,
blood glucose level measurements at a plurality of time points over the third period, and
insulin intake data at a plurality of time points over the third period;
generating predicted blood glucose levels at a plurality of time points over a first time interval of the third period using the personalized blood glucose regulation model and the third plurality of data sets;
creating a second training data set, a second validation data set, and a second test data set using the third plurality of data sets and the predicted blood glucose levels at the plurality of time points over the first time interval;
training a machine learning model using the second training data set and the second validation data set;
determining a second evaluation metric of the machine learning model using the second test data set;
generating a personalized hybrid model comprising the personalized blood glucose regulation model and the machine learning model; and
determining the personalized hybrid model as the working personalized blood glucose regulation model.

22. The method of claim 15, further comprising providing a simulated glucose curve over a period to the individual via a user interface based on the predicted blood glucose levels.

23. The method of claim 22, further comprising identifying a risk of glucose excursion to the individual via the user interface based on the simulated glucose curve.

24. The method of claim 22, further comprising providing a recommendation of insulin molecules and doses for bolus insulin intake and basal insulin intake to the individual via the user interface based on the simulated glucose curve.

25. The method of claim 22, further comprising providing a recommendation of a nutritional plan and/or a physical activity plan to the individual via the user interface based on the simulated glucose curve.

26. The method of claim 22, wherein providing an instruction for regulating the individual's blood glucose level comprises providing a corrective recommendation to the individual via a user interface based on the simulated glucose curve.

27. The method of claim 23, further comprising
identifying a risk of hypoglycemia excursion; and
in response to the identified risk of hypoglycemia excursion, sending an alert or a command to a vehicle, a driver safety system, or a driving command center or network.
